(19) United States
(12) Patent Application Publication
(10) Pub. No.: US 2025/0000764 A1
(43) Pub. Date: Jan. 2, 2025

(54) HAIR TREATMENT COMPOSITION WITH FATTY ALCOHOL, WAX, AND SILICONE

(71) Applicant: L'OREAL, Paris (FR)

(72) Inventors: Kazumitsu KAWAKAMI, Westfield, NJ (US); Rachel N. MAGEE, Westfield, NJ (US); Liliana XAVIER, Elizabeth, NJ (US)

(21) Appl. No.: 18/343,313

(22) Filed: Jun. 28, 2023

(51) Int. Cl. A61K 8/34; A61K 8/41; A61K 8/73; A61K 8/896; A61K 8/92; A61Q 5/00

(52) U.S. Cl. CPC A61K 8/342; A61K 8/416; A61K 8/73; A61K 8/896; A61K 8/92; A61Q 5/002

(57) ABSTRACT

The instant disclosure is drawn to hair treatment compositions; and to methods for conditioning, managing, and improving the look and feel of hair. The compositions include: (a) one or more fatty alcohols; (b) one or more mono-alkyl cationic surfactants; (c) one or more di-alkyl cationic surfactants; (d) one more fatty compounds other than the fatty alcohols of (a), wherein the one more fatty compounds other than the fatty alcohols of (a) provide at least 1 wt. % of one or more fatty ester waxes to the hair treatment composition, based on the total weight of the hair treatment composition; (e) one or more silicone oils; (f) one or more amino-functionalized silicones; and (g) water. The compositions provide deep conditioning and other benefits to hair.

### FIELD OF THE DISCLOSURE

The present disclosure relates to hair treatment compositions containing a fatty alcohol, a wax, and a silicone; and to methods for treating hair, for example, conditioning, managing, and improving the look and feel of hair.

### BACKGROUND

Many individuals suffer from dry and damaged hair. Dryness and damage can occur due to several factors including weather exposure, mechanical treatments (e.g. brushing hair), excessive treatments using chemicals, dying hair, heat styling, etc. In combination, using cleansing products that can be excessively stripping of hair's natural oils can also not only lead to split ends and dull hair, but also exacerbate dry hair. To mitigate the damage, oil treatments, conditioner, hair masks, and chemical treatments are commonly used.

The popularity and usage of oils for dry hair treatments has increased due to their effectiveness and simplicity. Commonly used oils include olive oil, mineral oil, avocado oil, apricot kernel oil, rice bran oil, and coconut oil. However, one problem is that effects are not usually seen after more than several hours (e.g. 8 hours) of treatment and several treatments are usually required, making it time consuming and labor intensive.

Individuals desire a treatment for hair or damaged hair that is not time consuming and labor intensive to use. A variety of approaches have been developed to condition the hair. A common method of providing conditioning benefit is through using conditioning agents such as cationic surfactants and polymers, high melting point fatty compounds, low melting point oils, silicone compounds, and combinations thereof. Most of these conditioning agents are known to provide various conditioning benefits.

However, there is still a need for providing improved hair manageability, for example, improved hair alignment, reduced unwanted volume (especially reduced frizz), and increased shine. There is also a need to develop hair care products that can impart other benefits at the same time in addition to caring and conditioning benefits, such as styling, volume, shaping, curl definition (for curly or wavy hair), and restyle-ability or reshaping (without the need to reapply the product).

### SUMMARY OF THE DISCLOSURE

The instant disclosure is drawn to a hair treatment composition and to methods for conditioning, managing, and improving the look and feel of hair. The compositions are useful in methods for treating hair, for example, conditioning, smoothing, reducing frizz and “fly away,” and improving ease of detangling and volume effects. The compositions include a multitude of ingredients providing synergistic blends of improvements to hair. However, the more complex a formulation becomes, the more difficult it is to avoid negative interactions between components. Major problems include compatibility and stability, especially long-term stability. Often, inclusion of one ingredient for a specific purpose will negatively impact or eliminate the beneficial properties of another component, included for a different purpose. The inventors of the instant disclosure, however, discovered compositions that effectively incorporate a plurality of beneficial components into a single composition, which is stable and effectively and simultaneously imparts a myriad of benefits to the hair. For example, the composition imparts deep conditioning, smoothness, shine/gloss to the hair in addition to reducing instances of “fly away.” The conditioning effects also improve the slip of the hair even when the hair is wet, which eases detangling of hair.

The compositions typically include:
(a) one or more fatty alcohols
(b) one or more mono-alkyl cationic surfactants;
(c) one or more di-alkyl cationic surfactants;
(d) one or more fatty compounds other than the fatty alcohols of (a),
   wherein at least one of the one or more fatty compounds is wax in an amount of at least 1 wt. %, based on the total weight of the hair treatment composition;
(e) one or more silicone oils; and
(f) one or more amino-functionalized silicones; and
(g) water,
   wherein all weight percentages are based on the total weight of the hair treatment composition.

In various embodiments, the composition further includes: one or more water-soluble solvents; one or more thickening agents; one or more amphoteric surfactants, and/or nonionic surfactants; and/or one or more miscellaneous ingredients selected from amino acids, pH adjusters, salts, fragrances, preservatives, antioxidants, composition colorants, botanical extracts, proteins, peptides, particulate fillers, vitamins, or a combination thereof.

Nonlimiting examples of useful fatty alcohols include linear or branched fatty alcohols having from 10 to 30 carbon atoms, preferably from 12 to 28 carbon atoms. In various embodiments, the fatty alcohols are selected from capryl alcohol, pelargonic alcohol, decyl alcohol, undecyl alcohol, lauryl alcohol, tridecyl alcohol, myristyl alcohol, pentadecyl alcohol, cetyl alcohol, palmitoleyl alcohol, isostearyl alcohol, isocetyl alcohol, heptadecyl alcohol, stearyl alcohol, cetearyl alcohol, oleyl alcohol, nonadecyl alcohol, arachidyl alcohol, heneicosyl alcohol, behenyl alcohol, erucyl alcohol, lignoceryl alcohol, ceryl alcohol, 1-heptacosanol, montanyl alcohol, 1-nonacosanol, and myricyl alcohol. In addition, the one or more fatty alcohols may be selected from decyl alcohol, undecyl alcohol, dodecyl, myristyl, cetyl alcohol, stearyl alcohol, cetearyl alcohol, isostearyl alcohol, isocetyl alcohol, behenyl alcohol, linalool, oleyl alcohol, myricyl alcohol and a combination thereof. In some instances, the cosmetic compositions preferably include cetyl alcohol, behenyl alcohol, cetearyl alcohol, or a combination thereof. Furthermore, nonlimiting fatty alcohols include C14-22 linear fatty alcohols.

Nonlimiting examples of mono-alkyl cationic surfactants include mono-alkyl trimonium halide compounds, preferably, one or more mono-alkyl trimonium halide compounds selected from cetrimonium chloride, steartrimonium chloride, behentrimonium chloride, cocotrimonium chloride, cocamidopropyltrimonium chloride, or a combination thereof.

Nonlimiting examples of di-alkyl dimonium cationic surfactants include, but are not limited to di-alkyl dimonium halide compounds, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, dicetyl dimethyl ammonium chloride, dicetyldimonium chloride, dicetyldimonium bromide, or a combination thereof.

The amount of the one or more mono-alkyl cationic surfactants is typically higher than the amount of the one or more di-alkyl dimonium cationic surfactants, for example, the amount of the one or more mono-alkyl cationic surfactant may be double or triple the amount of the one or more di-alkyl cationic surfactants. Furthermore, the amount of the one or more fatty alcohols is typically higher than the combined amount of the mono-alkyl- and di-alkyl-cationic surfactant.

Nonlimiting examples of fatty ester waxes include hydrogenated castor oil wax, candelilla wax, carnauba wax, hydrogenated jojoba wax, beeswax, ozokerite, lanolin wax, montan wax, paraffin, palm kernel wax, ceresin, silicone waxes, cetyl esters, or a combination thereof.

Nonlimiting examples of silicone oils include dimethicone, dimethiconol, cyclomethicone, polysilicone-11, phenyl trimethicone, or a combination thereof. Amino-functionalized silicones are distinguished from silicone oils, although they are a type of silicone oil. Nonlimiting examples of amino-functionalized silicones include amodimethicone, bis-hydroxy/methoxy amodimethicone, bis-cetearyl amodimethicone, bis(C13-15 alkoxy) PG amodimethicone, aminopropyl phenyl trimethicone, aminopropyl dimethicone, bis-amino PEG/PPG-41/3 aminoethyl PG-propyl dimethicone, or a combination thereof. Typically, a combination of one or more silicone oils and one or more amino-functionalized silicones is used.

As the name suggests, water-soluble solvents are compounds which combine with water and are soluble or miscible to an extent with water. Nonlimiting examples include glycerin, C1-C6 mono-alcohols, polyols (polyhydric alcohols), glycols, or a combination thereof. In some instances, it can be beneficial to include more than one water soluble solvent, i.e., a blend of two or more water solvents.

Thickening agents are useful for thickening the hair treatment composition and contribute to a pleasant viscosity, form, and feel of the composition. Thickening agents include anionic, nonionic, and cationic thickening agents, typically thickening polymers. In some instances, use of one or more nonionic thickening polymers is useful, for example nonionic polysaccharide thickening agents. Nonlimiting examples of nonionic polysaccharide thickening agents include gums, such as sclerotium gum, guar gums, guar gum derivatives, and xanthan gum, celluloses, such as hydroxyalkyl celluloses optionally modified with a hydrophobic group, inulins, carrageenans, carrageenan derivatives, or a combination thereof.

The hair treatment compositions typically include one or more surfactants, for example, one or more amphoteric surfactants, nonionic surfactants, cationic surfactants other than the one or more mono-alkyl cationic surfactants (b) and the one or more di-alkyl cationic surfactants of (c), or a combination thereof.

Miscellaneous ingredients may also be included in the hair treatment compositions. Nonlimiting examples include amino acids, pH adjusters, salts, fragrances, preservatives, antioxidants, composition colorants, botanical extracts, proteins, peptides, particulate fillers, vitamins, or a combination thereof.

The compositions can be applied immediately after shampooing the hair, for example, in place of a conditioner. The compositions can also be applied on hair immediately after shampooing and conditioning the hair, for example, as a mask rinse-off or leave-in treatment. The compositions can also be applied before shampooing the hair as pre-treatment compositions. In various embodiments, after the compositions are applied to the hair, the hair is rinsed with water, dried, and styled as desired. In other embodiments, the compositions are applied as a leave-on product. For example, the compositions can be applied to wet or damp hair and allowed to remain on the hair indefinitely, i.e., the hair composition is not removed or rinsed from the hair prior to styling the hair.

### DETAILED DESCRIPTION OF THE DISCLOSURE

The hair treatment compositions of the instant disclosure are particularly useful for conditioning hair, for example, by providing smoothing, reducing frizz and “fly away,” and improving ease of detangling. Despite containing disparate types of ingredients, including ingredients that are difficult to formulate and use with one another, the compositions are stable, and the benefits provided by various ingredients are maintained. The stability and effectiveness of the compositions is due, at least in part, to the combination of certain types, amounts, and ratios. The penetrative ability of the compositions allows for conditioning actives (such as cationic surfactants) and fatty compounds (including fatty alcohols and oils) to embed deep into the hair shaft preventing them from being easily removed by subsequent washing and rinsing. The treated hair does not suffer from being “weighed down” nor does it appear “greasy,” which is a common problem with typical conditioning compositions. Rather, the hair is light and manageable with a healthy and natural shine. The compositions typically include:
(a) 2 to 9 wt. % of one or more fatty alcohols
(b) 2 to 4 wt. % of one or more mono-alkyl cationic surfactants;
(c) 0.1 to 3 wt. % of one or more di-alkyl cationic surfactants;
(d) 0.5 to 10 wt. % of one or more fatty compounds other than the fatty alcohols of (a),
   wherein the one or more fatty compounds other than the fatty alcohols of (a) provide at least 1 wt. % of one or more fatty ester waxes to the hair treatment composition, based on the total weight of the hair treatment composition;
(e) 1 to 5 wt. % of one or more silicone oils; and
(f) 0.5 to 5 wt. % of one or more amino-functionalized silicones; and
(g) 60 to 90 wt. % water,
   wherein all weight percentages are based on a total weight of the hair treatment composition.

The compositions are typically oil-in-water emulsions or dispersions, having a creamy or lotion-like consistency that is pleasant to consumers and easy to use. The compositions typically have an opaque appearance, which can be enhanced, if desired, with pearlescent agents.

### (a) Fatty Alcohols

The term “fatty alcohol” means an alcohol comprising at least one hydroxyl group (OH), and comprising at least 8 carbon atoms, and which is neither oxyalkylenated (in particular neither oxyethylenated nor oxypropylenated) nor glycerolated. The fatty alcohols can be represented by: R—OH, wherein R denotes a saturated (alkyl) or unsaturated (alkenyl) group, linear or branched, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms.

In various embodiments, the compositions include at least one solid fatty alcohol. Solid fatty alcohols are fatty alcohols that are solid at ambient temperature and at atmospheric pressure (25° C., 780 mmHg), and are insoluble in water, i.e., they have a water solubility of less than 1% by weight, preferably less than 0.5% by weight, at 25° C., 1 atm. The solid fatty alcohols may be represented by: R—OH, wherein R denotes a linear alkyl group, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms. Nonlimiting examples include lauryl alcohol (1-dodecanol); myristyl alcohol (1-tetradecanol), cetyl alcohol (1-hexadecanol), stearyl alcohol (1-octadecanol), arachidyl alcohol (1-eicosanol), behenyl alcohol (1-docosanol), lignoceryl alcohol (1-tetracosanol), ceryl alcohol (1-hexacosanol), montanyl alcohol (1-octacosanol), myricylic alcohol (1-triacontanol), and combinations thereof. In a preferred embodiment, the compositions include at least one solid fatty alcohol selected from myristyl alcohol, cetyl alcohol, stearyl alcohol, behenyl alcohol and combinations thereof such as cetylstearyl or cetearyl alcohol.

In various embodiments, the compositions include at least one liquid fatty alcohol, in particular containing C10-C34 and preferably have branched carbon chains and/or have one or more, preferably 1 to 3 double bonds. They are preferably branched and/or unsaturated (C═C double bond) and contain from 12 to 40 carbon atoms. The liquid fatty alcohols may be represented by: R—OH, wherein R denotes a C12-C24 branched or straight alkyl group or an alkenyl group, R being optionally substituted by one or more hydroxy groups. In certain embodiments, the liquid fatty alcohols are selected from branched saturated alcohols. Preferably, R does not contain a hydroxyl group. Nonlimiting examples include oleyl alcohol, linoleyl alcohol, linolenyl alcohol, isocetyl alcohol, isostearyl alcohol, 2-octyl-1-dodecanol, 2-butyloctanol, 2-hexyl-1-decanol, 2-decyl-1-tetradecanol, 2-tetradecyl-1-cetanol and combinations thereof. In other embodiments, the compositions are free or essentially free from liquid fatty alcohols, including the liquid fatty alcohols referenced above.

In a preferred embodiment, the one or more fatty alcohols are linear (straight chain) saturated fatty alcohols having from 10 to 30 carbon atoms, preferably from 12 to 28 carbon atoms, more preferably from 14 to 24 carbon atoms. Nonlimiting examples include decyl alcohol, undecyl alcohol, dodecyl alcohol, myristyl alcohol, cetyl alcohol, stearyl alcohol, cetearyl alcohol, behenyl alcohol, myricyl alcohol and a combination thereof.

The total amount of the one or more fatty alcohols in the hair treatment composition will vary. Nonetheless, in various embodiments, the hair treatment composition includes about 2 to about 10 wt. % of the one or more fatty alcohols, based on a total weight of the hair treatment composition. In further embodiments, the hair treatment composition includes about 2 to about 9 wt. %, about 2 to about 8 wt. %, about 2 to about 7 wt. %, about 2.5 to about 10 wt. %, about 2.5 to about 8 wt. %, about 2.5 to about 7 wt. %, about 3 to about 10 wt. %, about 3 to about 9 wt. %, about 3 to about 8 wt. %, about 3 to about 7 wt. %, about 4 to about 10 wt. %, about 4 to about 9 wt. %, about 4 to about 8 wt. %, or about 4 to about 7 wt. % of the one or more fatty alcohols, based on a total weight of the hair treatment composition.

### Cationic Surfactants

The term “cationic surfactant” as defined by the instant disclosure is a surfactant that may be positively charged when it is contained in the hair treatment compositions according to the disclosure. The cationic surfactant may bear one or more positive permanent charges or may contain one or more functional groups that are cationizable in the composition according to the disclosure. The hair treatment compositions of the instant case include one more mono-alkyl cationic surfactants (b) and (c) one or more di-alkyl cationic surfactants. However, other cationic surfactants can also be included.

The total amount of the cationic surfactants in the hair treatment composition will vary. Nonetheless, in various embodiments, the hair treatment compositions include about 1 to about 10 wt. % of total cationic surfactants, based on a total weight of the composition. In further embodiments, the hair treatment composition includes about 1 to about 8 wt. %, about 1 to about 6 wt. %, about 1 to about 5 wt. %, about 1 to about 4 wt. %, about 1.5 to about 10 wt. %, 1.5 to about 8 wt. %, about 1.5 to about 6 wt. %, about 1.5 to about 5 wt. %, about 1.5 to about 4 wt. %, about 2 to about 10 wt. %, about 2 to about 8 wt. %, about 2 to about 6 wt. %, about 2 to about 5 wt. %, about 2 to about 4 wt. %, about 2.5 to about 10 wt. %, about 2.5 to about 8 wt. %, about 2.5 to about 6 wt. %, about 2.5 to about 5 wt. %, or about 2.5 to about 4 wt. % of total cationic surfactants, based on a total weight of the hair treatment composition.

### (b) Mono-Alkyl Cationic Surfactants

Mono-alkyl cationic surfactants useful herein are primary, secondary, and tertiary amines having one long alkyl or alkenyl group of from about 12 to about 30 carbon atoms, preferably from 16 to 24 carbon atoms, more preferably from 18 to 22 alkyl group. For example, mono-alkyl cationic surfactants include mono-alkyl trimonium halide compounds. Nonlimiting examples of mono-alkyl trimonium halide compounds include cetrimonium chloride, steartrimonium chloride, behentrimonium chloride, cocotrimonium chloride, cocamidopropyltrimonium chloride. Preferred are cetrimonium chloride, steartrimonium chloride and behentrimonium chloride.

In various embodiments, the hair treatment compositions include behentrimonium chloride, cetrimonium chloride, or a combination thereof.

Mono-alkyl cationic surfactants also include mono-alkyl amidoamines. Particularly useful are tertiary amidoamines having an alkyl group of from about 12 to about 22 carbon atoms, preferably from about 16 to about 22 carbon atoms. Exemplary tertiary amido amines include: stearamidopropyldimethylamine, stearamidopropyl-diethylamine, stearamidoethyldiethylamine, stearamidoethyldimethylamine, palmitamidopropyldimethylamine, palmitamidopropyldiethylamine, palmitamidoethyl-diethylamine, palmitamidoethyldimethylamine, behenamidopropyldimethylamine, behenamidopropyldiethylamine, behenamidoethyldiethylamine, behenamidoethyl-dimethylamine, arachidamidopropyldimethylamine, arachidamidopropyldiethylamine, arachidamidoethyldiethylamine, arachidamidoethyldimethylamine, diethylaminoethyl-stearamide, and a combination thereof.

The total amount of the one or more mono-alkyl cationic surfactants will vary. Nonetheless, in various embodiments, the hair treatment compositions include about 1 to about 5 wt. % of the one or more mono-alkyl cationic surfactants, based on a total weight of the hair treatment composition. In further embodiments, the hair treatment composition includes about 1 to about 4.5 wt. %, about 1 to about 4 wt. %, about 1 to about 3.5 wt. % about 1 to about 3 wt. %, about 1.5 to about 5 wt. %, about 1.5 to about 4.5 wt. %, about 1.5 to about 4 wt. %, about 1.5 to about 3 wt. %, about 2 to about 5 wt. %, about 2 to about 4.5 wt. %, about 2 to about 4 wt. %, about 2 to about 3.5 wt. %, about 2 to about 3 wt. %, about 2.2 to about 5 wt. %, about 2.2 to about 4.5 wt. %, about 2.2 to about 4 wt. %, about 2.2 to about 3.5 wt. %, or about 2.2 to about 3 wt. % of the one or more mono-alkyl cationic surfactants, based on a total weight of the hair treatment composition.

### (c) Di-Alkyl Cationic Surfactants

Di-alkyl cationic surfactants includes those of formula (I) and salts thereof:

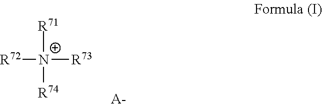

wherein two of R⁷¹, R⁷², R⁷³, and R⁷⁴ are selected from an aliphatic group of from 12 to 30 carbon atoms, preferably from 16 to 24 carbon atoms, more preferably from 18 to 22 carbon atoms or an aromatic, alkoxy, polyoxyalkylene, alkylamido, hydroxyalkyl, aryl or alkylaryl group having up to about 30 carbon atoms;
the remainder of R⁷¹, R⁷², R⁷³ and R⁷⁴ are independently selected from an aliphatic group of from 1 to about 8 carbon atoms, preferably from 1 to 3 carbon atoms or an aromatic, alkoxy, polyoxyalkylene, alkylamido, hydroxyalkyl, aryl or alkylaryl group having up to about 8 carbon atoms; and
A⁻ is an anion, for example, a halide, such as chloride or bromide, a C1-C4 alkyl sulfate such as methosulfate and ethosulfate, and combinations thereof.

The aliphatic groups for Formula (I) can contain, in addition to carbon and hydrogen atoms, ether linkages, and other groups such as amino groups. The longer chain aliphatic groups, e.g., those of about 16 carbons, or higher, can be saturated or unsaturated. Preferably, two of R⁷¹, R⁷², R⁷³ and R⁷⁴ are selected from an alkyl group of from 12 to 30 carbon atoms, preferably from 16 to 24 carbon atoms, more preferably from 18 to 22 carbon atoms; and the remainder of R⁷¹, R⁷², R⁷³ and R⁷⁴ are independently selected from CH₃, C₂H₅, C₂H₄OH, CH₂C₆H₅, and combinations thereof.

Nonlimiting examples of di-alkyl cationic surfactants of Formula (I) include dialkyl (14-18) dimethyl ammonium chloride, ditallow alkyl dimethyl ammonium chloride, dihydrogenated tallow alkyl dimethyl ammonium chloride, distearyl dimethyl ammonium chloride, dicetyl dimethyl ammonium chloride, dicetyldimonium chloride, dicetyldimonium bromide, and a combination thereof.

In various embodiments, the one or more di-alkyl cationic surfactants are selected from di-alkyl dimonium halide compounds. Nonlimiting examples include dialkyl (14-18) dimethyl ammonium chloride, ditallow alkyl dimethyl ammonium chloride, dihydrogenated tallow alkyl dimethyl ammonium chloride, distearyl dimethyl ammonium chloride, dicetyl dimethyl ammonium chloride, dicetyldimonium chloride, dicetyldimonium bromide, and a combination thereof. In a preferred embodiment, the di-alkyl dimonium halide compounds are selected from dicetyldimonium chloride, dicetyldimonium bromide, and a combination thereof. In a preferred embodiment, the hair treatment composition includes dicetyldimonium chloride.

The total amount of the one or more di-alkyl cationic surfactants will vary. Nonetheless, in various embodiments, the hair treatment compositions include about 0.1 to about 5 wt. % of the one or more di-alkyl cationic surfactants, based on a total weight of the hair treatment composition. In further embodiments, the hair treatment composition includes about 0.1 to about 4 wt. %, about 0.1 to about 3 wt. %, about 0.1 to about 2 wt. %, about 0.1 to about 1 wt. %, about 0.5 to about 5 wt. %, 0.5 to about 4 wt. %, about 0.5 to about 3 wt. %, about 0.5 to about 2 wt. %, or about 0.5 to about 1 wt. %, based on a total weight of the hair treatment composition.

### Ratio of (b) to (c)

The weight ratio of the one or more mono-alkyl cationic surfactants of (b) to the one or more di-alkyl cationic surfactants of (c) will vary. Nonetheless, the hair treatment compositions typically include a higher amount of the one or more mono-alkyl cationic surfactants of (a) than the one or more di-alkyl cationic surfactants of (b). In various embodiments, the one or more mono-alkyl cationic surfactants of (b) and the one or more di-alkyl cationic surfactants of (c) are in a weight ratio of about 2:1 to about 10:1 ((b):(c)). In further embodiments, the weight ratio is about 2:1 to about 8:1, about 2:1 to about 6:1, about 2:1 to about 5:1, about 2:1 to about 4:1, about 3:1 to about 10:1, about 3:1 to about 8:1, about 3:1 to about 6:1, about 3:1 to about 5:1, about 3:1 to about 4:1.

Ratio of (a) to ((b)+(c))

The weight ratio of the one or more fatty alcohols of (a) to the combined amount of the one or more mono-alkyl cationic surfactants of (b) and the one or more di-alkyl cationic surfactants of (c) will vary. Typically, however, the amount of the one or more fatty alcohols of (a) is at least equal to or greater than the combined amount of the one or more mono-alkyl cationic surfactants of (b) and the one or more di-alkyl cationic surfactants of (c). For example, in various embodiments, the weight ratio of the one or more fatty alcohols of (a) to the combined amount of the one or more mono-alkyl cationic surfactants of (b) and the one or more di-alkyl cationic surfactants of (c) is about 1:1 to about 2.4:1. In further embodiments, the weight ratio is about 1:1 to about 2.3:1, about 1:1 to about 2.2:1, about 1:1 to about 2.1:1, about 1:1 to about 2:1, greater than 1:1 to about 2.4:1, greater than 1:1 to about 2.3:1, greater than 1:1 to about 2.2:1, greater than 1:1 to about 2.1:1, greater than 1:1 to about 2:1, about 1.2:1 to about 2.4:1, 1.2:1 to about 2.3:1, about 1.2:1 to about 2.2:1, about 1.2:1 to about 2.1:1, about 1.2:1 to about 2:1, 1.5:1 to about 2.4:1, about 1.5:1 to about 2.3:1, about 1.5:1 to about 2.2:1, about 1.5:1 to about 2:1, about 1.8:1 to about 2.4:1, about 1.8:1 to about 2.3:1, about 1.8:1 to about 2.2:1, about 1.8:1 to about 2.1:1, or about 1.8:1 to about 2:1.

(d) Fatty Compounds Other than the Fatty Alcohols of (a)

For purposes of the instant disclosure “fatty compounds other than the fatty alcohol of (a)” is understood to be non-silicone fatty compounds that do not include the fatty alcohols of (a). The term “fatty compound” is interchangeable with the term “fatty substance,” and means an organic compound without silicone that is insoluble in water at ordinary temperature (25° C.) and at atmospheric pressure (760 mmHg), i.e., which has a solubility of less than 5%, preferably less than 1% and even more preferentially less than 0.1%. They have in their structure a hydrocarbon-based chain containing at least 6 carbon atoms.

Nonlimiting examples of fatty compounds include C6-C16 hydrocarbons, hydrocarbons containing more than 16 carbon atoms, oils animal origin, oil of plant origin, hydrocarbon-based oils, synthetic triglycerides, fluoro oils, non-salified fatty acids, fatty acid and/or fatty alcohol esters, waxes, and combinations thereof. Fatty compounds may be classified as fatty ester, fatty ethers, propylene glycol fatty acid esters, fatty carbonate esters, oils, and waxes. However, the one or more fatty compounds of the instant disclosure are not limited to these classes of fatty compounds.

i. Fatty Esters

Non-limiting examples of fatty esters include fatty esters from a C₆-C₃₂ fatty acid and/or a C₆-C₃₂ fatty alcohol. These esters may be esters of saturated or unsaturated, linear or branched C₁-C₂₆ aliphatic mono or polyacids and of saturated or unsaturated, linear or branched C₁-C₂₆ aliphatic mono or polyalcohols, the total number of carbon atoms in the esters being greater than or equal to 10. In some cases, for the esters of monoalcohols, at least one of the alcohol or the acid from which the esters of the invention result is branched. Among the monoesters of monoacids and of monoalcohols, mention may be made of ethyl palmitate, isopropyl palmitate, alkyl myristates such as isopropyl myristate or ethyl myristate, isocetyl stearate, 2-ethylhexyl isononanoate, isononyl isononanoate, isodecyl neopentanoate, and isostearyl neopentanoate.

In various embodiments, the compositions of the instant disclosure may include cetyl esters. Cetyl esters are a mixture of the following esters of saturated fatty acids and fatty alcohols: cetyl palmitate, cetyl stearate, myristyl myristate, myristyl stearate, cetyl myristate, and stearyl stearate.

Mention is made of esters of C₄-C₂₂ dicarboxylic or tricarboxylic acids and of C₁-C₂₂ alcohols and esters of monocarboxylic, dicarboxylic or tricarboxylic acids and of C₄-C₂₆ dihydroxy, trihydroxy, tetrahydroxy or pentahydroxy non-sugar alcohols may also be used. Mention may be made in particular of diethyl sebacate, diisopropyl sebacate, diisopropyl adipate, di-n-propyl adipate, triisopropyl citrate, glyceryl trilactate, glyceryl trioctanoate, neopentyl glycol diheptanoate, and diethylene glycol diisononanoate.

Nonlimiting examples of liquid esters (ester oils) or liquid fatty esters that may be mentioned include, for example, sunflower oil, corn oil, soybean oil, marrow oil, grapeseed oil, sesame seed oil, hazelnut oil, apricot oil, macadamia oil, arara oil, castor oil, avocado oil, olive oil, rapeseed oil, coconut oil, wheatgerm oil, sweet almond oil, apricot oil, safflower oil, candlenut oil, coconut oil, camellina oil, tamanu oil, babassu oil and pracaxi oil, jojoba oil, and shea butter oil, and caprylic/capric triglyceride.

Nonlimiting examples of solid fatty esters include solid esters obtained from C₉-C₂₆ fatty acids and from C₉-C₂₆ fatty alcohols. Among these esters, mention may be made of octyldodecyl behenate, isocetyl behenate, cetyl lactate, stearyl octanoate, octyl octanoate, cetyl octanoate, decyl oleate, myristyl stearate, octyl palmitate, octyl pelargonate, octyl stearate, alkyl myristates such as cetyl myristate, myristyl myristate or stearyl myristate, and hexyl stearate.

In a preferred embodiment, at least one of the one or more emollients is selected from cetyl esters, purcellin oil (cetearyl octanoate), isopropyl myristate, isopropyl palmitate, C12-C15 alkyl benzoate, 2-ethylphenyl benzoate, isopropyl lanolate, hexyl laurate, diisopropyl adipate, isononyl isononanoate, oleyl erucate, 2-ethylhexyl palmitate, isostearyl isostearate, diisopropyl sebacate, octanoates, decanoates or ricinoleates of alcohols or polyalcohols, hydroxylated esters, pentaerythritol esters, diisostearyl malate, neopentyl glycol dioctanoate, dibutyl sebacate, di-C12-13 alkyl malate, dicetearyl dimer dilinoleate, dicetyl adipate, diisocetyl adipate, diisononyl adipate, diisostearyl dimer dilinoleate, diisostearyl fumarate, and a combination thereof.

The esters of fatty acids and/or of fatty alcohols, are esters of saturated or unsaturated, linear or branched C1-C26 aliphatic mono- or polyacids and of saturated or unsaturated, linear or branched C1-C26 aliphatic mono- or polyalcohols, the total carbon number of the esters more particularly being greater than or equal to 10. Among the monoesters, mention may be made of dihydroabietyl behenate; octyldodecyl behenate; isocetyl behenate; cetyl lactate; C12-C15 alkyl lactate; isostearyl lactate; lauryl lactate; linoleyl lactate; oleyl lactate; (iso) stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate; dioctyl malate, hexyl laurate, 2-hexyldecyl laurate. Still within the context of this variant, esters of C4-C22 dicarboxylic or tricarboxylic acids and of C1-C22 alcohols and esters of mono-, di- or tricarboxylic acids and of C2-C26 di-, tri-, tetra- or pentahydroxy alcohols may also be used.

ii. Fatty Ethers

Nonlimiting examples of fatty ethers include olyoxyethylene cetyl/stearyl ether, polyoxyethylene cholesterol ether, polyoxyethylene laurate or dilaurate, polyoxyethylene stearate or distearate, polyoxyethylene lauryl or stearyl ether, dicaprylyl ether, dicetyl ether distearyl ether, dodecyl ether, dilauryl ether, dimyristyl ether, diisononyl ether, or a combination thereof. Non-limiting examples of suitable polyoxyethylene fatty ethers include, but are not limited to, polyoxyethylene cetyl/stearyl ether, polyoxyethylene cholesterol ether, polyoxyethylene laurate or dilaurate, polyoxyethylene stearate or distearate, polyoxyethylene lauryl or stearyl ether, and combinations thereof, wherein the polyoxyethylene head group ranges from about 2 to about 100 groups. In certain embodiments, the polyoxyethylene fatty ethers include polyoxyethylene stearyl ether, polyoxyethylene myristyl ether, polyoxyethylene lauryl ether having from about 3 to about 10 oxyethylene units and combinations thereof. In yet another embodiment, at least one of the emollients is a fatty ether selected from stearyl ether, dicaprylyl ether, dicetyl ether distearyl ether, dodecyl ether, dilauryl ether, dimyristyl ether, diisononyl ether, or a combination thereof.

iii. Propylene Glycol Fatty Acid Esters

Nonlimiting examples of propylene glycol fatty acid esters include propylene glycol esters of medium chain fatty acids (fatty acids having from 6 to 12 carbon atoms), such as propylene glycol dicaprylate/dicaprate, propylene glycol dipelargonate, and propylene glycol dilaurate. A preferred propylene glycol fatty acid ester is propylene glycol dicaprylate/dicaprate. The term “propylene glycol dicaprylate/dicaprate” is understood by those in the art to refer to a combination containing propylene glycol dicaprylate, propylene glycol dicaprylate-caprate, and propylene glycol dicaprate, which may vary in the ratio of these components. An example of a commercially available form of propylene glycol dicaprylate/dicaprate is “CAPTEX® 200,” available from the Abitec Corp. (Columbus, OH, USA).

iv. Fatty Carbonate Esters

Nonlimiting examples of fatty carbonate esters include dialkyl carbonates of formula: R₁O(C═O) R₂, wherein R₁ and R₂ are independently linear or branched, saturated or unsaturated alkyl chains having 1 to 30 carbon atoms, or having 2 to 28 carbon atoms, or having 4 to 25 carbon atoms, or having 6 to 22 carbon atoms, preferably one or more fatty carbonates selected from C14-15 dialkyl carbonate, dicaprylyl carbonate, diethyl carbonate, dihexyl carbonate, diethylhexyl carbonate, dimethoxyphenyl phenyloxoethyl ethylcarbonate, dimethyl carbonate, dipropyl carbonate, dipropylheptyl carbonate, dioctyl carbonate, and a combination thereof.

iv. Oils

The term “oil” means any fatty substance that is in liquid form at room temperature (20-25° C.) and at atmospheric pressure. The one or more oils can be hydrocarbon-based oils, fluoro oils, non-fluoro oils, or combinations thereof. The term “hydrocarbon-based oil” is interchangeable with the term “hydrocarbon oil.” The one or more oils can be “volatile oils” or “non-volatile oils.” For the purposes of the instant disclosure, the term “volatile oil” means an oil (or non-aqueous medium) capable of evaporating on contact with the skin in less than one hour, at room temperature and at atmospheric pressure. The volatile oil is a volatile cosmetic oil, which is liquid at room temperature, especially having a non-zero vapour pressure, at room temperature and atmospheric pressure, in particular having a vapour pressure ranging from 0.13 Pa to 40 000 Pa (10-3 to 300 mmHg), preferably ranging from 1.3 Pa to 13 000 Pa (0.01 to 100 mmHg), and preferentially ranging from 1.3 Pa to 1300 Pa (0.01 to 10 mmHg).

Suitable oils include, but are not limited to, natural oils, such as coconut oil; hydrocarbons, such as mineral oil and hydrogenated polyisobutene; fatty alcohols, such as octyldodecanol; esters, such as C₁₂-C₁₅ alkyl benzoate; diesters, such as propylene dipelarganate; and triesters, such as glyceryl trioctanoate. Non-limiting examples of oils that may, optionally, be included in the hair treatment compositions include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, or combinations of octyldodecanol, acetylated lanolin alcohol, cetyl acetate, isododecanol, polyglyceryl-3-diisostearate, castor oil, lanolin and lanolin derivatives, triisocetyl citrate, sorbitan sesquioleate, C₁₀-C₁₈ triglycerides, caprylic/capric/triglycerides, coconut oil, corn oil, cottonseed oil, glyceryl triacetyl hydroxystearate, glyceryl triacetyl ricinoleate, glyceryl trioctanoate, hydrogenated castor oil, linseed oil, mink oil, olive oil, palm oil, illipe butter, rapeseed oil, soybean oil, sunflower seed oil, tallow, tricaprin, trihydroxystearin, triisostearin, trilaurin, trilinolein, trimyristin, triolein, tripalmitin, tristearin, walnut oil, wheat germ oil, cholesterol, or combinations thereof.

The triglyceride oils of plant or synthetic origin are preferably chosen from liquid fatty acid triglycerides comprising from 6 to 30 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, castor oil, avocado oil, caprylic/capric acid triglycerides, for instance those sold by the company Stearineries Dubois or those sold under the names “Miglyol® 810,” “Miglyol® 812,” and “Miglyol® 818” by the company Dynamit Nobel, jojoba oil and shea butter oil.

The fluoro oils may be chosen from perfluoromethylcyclopentane and perfluoro-1,3-dimethylcyclohexane, sold under the names “Flutec® PC1” and “Flutec® PC3” by the company, BNFL Fluorochemicals; perfluoro-1,2-dimethylcyclobutane; perfluoroalkanes such as dodecafluoropentane and tetradecafluorohexane, sold under the names “PF 5050®” and “PF 5060®” by the company, 3M, or bromoperfluorooctyl sold under the name “Foralkyl®” by the company, Atochem; nonafluoromethoxybutane and nonafluoroethoxyisobutane; perfluoromorpholine derivatives such as 4-trifluoromethyl perfluoromorpholine sold under the name “PF 5052®” by the company, 3M.

### V. Waxes

Waxes are solids at room temperature and typically have a melting point greater than 30° C. or greater than 30° C. to about 100° C. Natural waxes include animal, vegetable/plant, mineral, or petroleum derived waxes. They are typically esters of fatty acids and long chain alcohols. Wax esters are derived from a variety of carboxylic acids and a variety of fatty alcohols.

Non-limiting examples of waxes include aliphatic esters, such as cetyl esters, stearyl esters, acacia, beeswax, ceresin, flower wax, citrus wax, carnauba wax, jojoba wax, Japanese wax, polyethylene, microcrystalline, rice bran, lanolin wax, mink, montan, bayberry, ouricury, ozokerite, palm kernel wax, paraffin, avocado wax, apple wax, shellac wax, salvia wax, candelilla wax, their polyalkylene glycol derivatives (e.g., PEG 6-20 beeswax or PEG-12 carnauba wax), and mixtures of any of the aforementioned waxes.

Additional nonlimiting examples of waxes include beeswax, hydrogenated alkyl olive esters (commercially available under the tradename “Phytowax® Olive”), carnauba wax, candelilla wax, ouricury wax, Japan wax, cork fibre wax or sugar cane wax, rice wax, montan wax, paraffin wax, lignite wax or microcrystalline wax, ceresin or ozokerite, palm kernel glycerides/hydrogenated palm glycerides and hydrogenated oils such as hydrogenated castor oil or jojoba oil, sugarcane, retamo, bayberry, rice bran, soy, castor, esparto, hydroxyoctacosanyl hydroxystearate, Chinese wax, cetyl palmitate, lanolin, shellac, and spermaceti; synthetic waxes such as those of the hydrocarbon type and polyethylene waxes obtained from the polymerization or copolymerization of ethylene, and Fischer-Tropsch® waxes, or else esters of fatty acids, such as octacosanyl stearate, glycerides which are solid at temperatures of above 30° C.

The hair treatment compositions of the instant disclosure typically include at least one wax. The at least one wax may be the predominant fatty compound of the one or more fatty compounds. In fact, the at least one wax may be in an amount greater than the total combined amounts of other fatty compounds (other than fatty alcohols) of (d), all other silicones of (e) and all other amino-functionalized silicones of (f). Typically, the hair treatment composition includes at least 1 wt. % of one or more waxes, based on the total weight of the hair treatment composition. In further embodiments, the hair treatment composition includes at least 1.1 wt. %, 1.2 wt. %, 1.3 wt. %, 1.4 wt. %, or 1.5 wt. % of one or more waxes, based on a total weight of the hair treatment compositions. For examples, in various embodiments, the hair treatment composition includes from at least 1 wt. % up to about 8 wt. % of one or more waxes. In further embodiments, the hair treatment composition includes at least 1.1 wt. % to about 8 wt. %, about 1.2 wt. % to about 8 wt. %, at least 1.3 to about 8 wt. %, at least 1.4 to about 8 wt. %, at least 1 wt. % to about 5 wt. %, at least 1.1 wt. % to about 5 wt. %, at least 1.2 to about 5 wt. %, at least 1.3 to about 5 wt. %, at least 1.4 to about 5 wt. %, at least 1.5 to about 5 wt. %, at least 1 to about 3.5 wt. %, at least 1.1 to about 3.5 wt. %, at least 1.2 to about 3.5 wt. %, at least 1.3 to about 3.5 wt. %, at least 1.4 to about 3.5 wt. %, or at least 1.5 to about 3.5 wt. % of one or more waxes, based on a total weight of the hair treatment composition.

The total amount of the one or more fatty compounds (including the one or more waxes) other than the one or more fatty alcohols of (a) will vary. Nonetheless, in various embodiments, the hair treatment composition includes about 1 to about 10 wt. % of the one or more fatty compounds, based on a total weight of the hair treatment composition. In further embodiments, the hair treatment composition includes about 1 to about 8 wt. %, about 1 to about 6 wt. %, about 1 to about 5 wt. %, about 1 to about 4 wt. %, about 1.5 to about 10 wt. %, about 1.5 to about 8 wt. %, about 1.5 to about 6 wt. %, about 1.5 to about 5 wt. %, about 1.5 to about 4 wt. %, about 2 to about 10 wt. %, about 2 to about 8 wt. %, about 2 to about 6 wt. %, about 2 to about 5 wt. %, or about 2 to about 4 wt. % of the one or more fatty compounds other than the fatty alcohol of (a), based on the total weight of the hair treatment composition.

### (e) Silicone Oils

In various embodiments, the leave-on hair treatment composition includes one or more silicone oils (also referred to as “silicone-based oils” or simply “silicones”). The one or more silicone oils can be volatile or non-volatile. Silicone oils include linear, branched, and cyclic silicone oils and include volatile and non-volatile silicone oils. Nonlimiting examples include polydimethylsiloxane (dimethicone), dimethiconol, phenyl-modified silicone, and blends of any of the foregoing. Additional nonlimiting examples include octamethylcyclotetrasiloxane, decamethylcyclopentasiloxane, dodecamethylcyclohexasiloxane, heptamethylhexyltrisiloxane, heptamethyl-octyltrisiloxane, hexamethyldisiloxane, octamethyltrisiloxane, decamethyltetrasiloxane and dodecamethylpentasiloxane, and combinations thereof. In a preferred embodiment, hair treatment composition includes at least one silicone oil selected from dimethicone, dimethiconol, or a combination thereof, preferably dimethicone.

The total amount of the one or more silicone oils in the hair treatment composition will vary. Nonetheless, in various embodiments the hair treatment composition includes about 0.1 to about 10 wt. % of one or more silicones. In further embodiments, the hair treatment composition includes about 0.1 to about 8 wt. %, about 0.1 to about 5 wt. %, about 0.1 to about 3 wt. %, about 0.5 to about 10 wt. %, about 0.5 to about 8 wt. %, about 0.5 to about 5 wt. %, about 0.5 to about 3 wt. %, about 1 to about 10 wt. %, about 1 to about 8 wt. %, about 1 to about 5 wt. %, or about 1 to about 3 wt. % of the one or more silicone oils, based on a total weight of the hair treatment composition.

### (f) Amino-Functionalized Silicones

The term “amino-functionalized silicone” or “amino silicones” means a silicone containing at least one primary amino, secondary amino, tertiary amino and/or quaternary ammonium group. The structure of the amino-functionalized silicone may be linear or branched, cyclic or non-cyclic. The amino functional group may be at any position in the silicone molecule, preferably at the end of the backbone (for example, in the case of amodimethicones) and/or in the side chain.

In some instances, an amino-functionalized silicone is selected from compounds having the following formula:

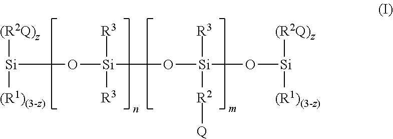

wherein each R¹ is independently selected from a C₁₋₃₀ alkyl group, a C₁₋₃₀ alkoxy group, a C₅₋₃₀ aryl group, a C₆₋₃₀ aralkyl group, a C₆₋₃₀ aralkyloxy group, a C₁₋₃₀ alkaryl group, a C₁₋₃₀ alkoxyaryl group, and a hydroxy group (preferably, each R¹ is independently selected from a C₁₋₃₀ alkyl group, a C₁₋₃₀ alkoxy group and a hydroxy group);
each R₂ is independently a divalent alkylene radical having one to ten carbon atoms (preferably, R² is a divalent alkylene radical having three to six carbon atoms);
each R³ is independently selected from a C₁₋₃₀ alkyl group, a C₅₋₃₀ aryl group, a C₆₋₃₀ aralkyl group and a C₁₋₃₀ alkaryl group (preferably, each R³ is independently selected from of a C₁₋₃₀ alkyl group);
Q is a monovalent radical selected from —NR⁴₂ and —NR⁴(CH₂)ₓNR⁴₂;
each R⁴ is independently selected from a hydrogen and a C₁₋₄ alkyl group;
x is 2 to 6;
z is 0 or 1;
n is 25 to 3,000 (preferably, 25 to 2,000; more preferably, 25 to 1,000; most preferably 25 to 500); and
m is 0 to 3,000 (preferably, 0 to 2,000; more preferably, 0 to 1,000; most preferably, 0 to 100);
with the proviso that at least 50 mol % of the total number of R¹ and R³ groups are methyl and with the proviso that when m is 0, z is 1.

Preferred R¹ groups include methyl, methoxy, ethyl, ethoxy, propyl, propoxy, isopropyl, isopropoxy, butyl, butoxy, isobutyl, isobutoxy, phenyl, xenyl, benzyl, phenylethyl, tolyl and hydoxy. Preferred R² divalent alkylene radicals include trimethylene, tetramethylene, pentamethylene, —CH₂CH(CH₃)CH₂ and CH₂CH₂CH(CH₃)CH₂.

Preferred R³ groups include methyl, ethyl, propyl, isopropyl, butyl, isobutyl, phenyl, xenyl, benzyl, phenylethyl and tolyl. Preferred R⁴ groups include methyl, ethyl, propyl, isopropyl, butyl and isobutyl. When z is 0, the amino-functionalized silicine has only pendant amine functional substituents in the polymer chain. When z is 1, the amino-functional silicone may have only terminal amine functional substituents (e.g., m=0) or may have both terminal and pendant amine functional substituents in the polymer chain (e.g., m>0). Preferably, n+m is 50 to 1,000. More preferably, n+m is 50 to 750. Still more preferably, n+m is 50 to 500. Most preferably, n+m is 50 to 250.

In some instances, the amino-functionalized silicones are alkoxylated and/or hydroxylated amino silicones. Suitable alkoxylated and/or hydroxylated amino silicones may be selected from compounds of the following formula:

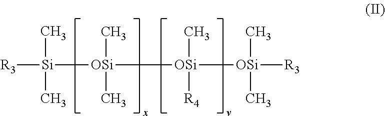

wherein R₃ is hydroxyl or OR₅, R₅ is a C₁ to C₄ alkyl group, R₄ is a group with structure according to the following formula:

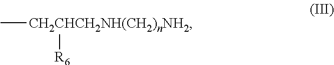

R₆ is a C₁ to C₄ alkyl, n is a 1 to 4, x is the same as “n” described above, and y is the same as “m” described above.

The silicone may be a polysiloxane corresponding to the following formula:

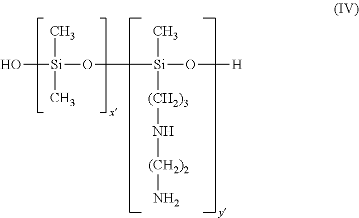

in which x′ and y′ are integers such that the weight-average molecular weight (Mw) is comprised between about 5000 and 500000;
b) amino silicones corresponding to following formula:

R′ₐG₃₋ₐ-Si(OSiG₂)ₙ-(OSiG_{b}R′_{2-b})ₘ—O-SiG₃₋ₐ-R′ₐ
in which:
G, which may be identical or different, designate a hydrogen atom, or a phenyl, OH or C₁-C₈ alkyl group, for example methyl, or C₁-C₈ alkoxy, for example methoxy,
a, which may be identical or different, denote the number 0 or an integer from 1 to 3, in particular 0;
b denotes 0 or 1, and in particular 1;
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
R′, which may be identical or different, denote a monovalent radical having formula —CqH₂qL in which q is a number ranging from 2 to 8 and L is an optionally quaternized amino group chosen from the following groups:

—NR″-Q-N(R″)₂

—N(R″)₂

—N+(R″)₃A-

—N+H(R″)₂A-

—N+H₂(R″)A-

—N(R″)-Q-N+R″H₂A-

—NR″-Q-N+(R″)₂HA-

—NR″-Q-N+(R″)₃A-,
in which R″, which may be identical or different, denote hydrogen, phenyl, benzyl, or a saturated monovalent hydrocarbon-based radical, for example a C₁-C₂₀ alkyl radical; Q denotes a linear or branched CrH₂ᵣ group, r being an integer ranging from 2 to 6, preferably from 2 to 4; and A-represents a cosmetically acceptable ion, in particular a halide such as fluoride, chloride, bromide, or iodide.

Another group of amino silicones corresponding to this definition is represented by silicones having the following formula:

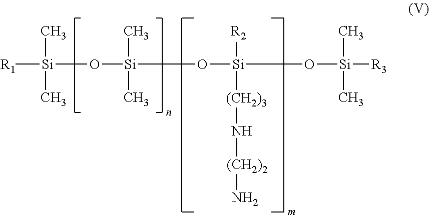

in which:
m and n are numbers such that the sum (n+m) can range from 1 to 1000, in particular from 50 to 250 and more particularly from 100 to 200, it being possible for n to denote a number from 0 to 999 and in particular from 49 to 249, and more particularly from 125 to 175, and for m to denote a number from 1 to 1000 and in particular from 1 to 10, and more particularly from 1 to 5;
R¹, R², R³, which may be identical or different, represent a hydroxy or C₁-C₄ alkoxy radical, where at least one of the radicals R¹ to R³ denotes an alkoxy radical.

The alkoxy radical is preferably a methoxy radical. The hydroxy/alkoxy mole ratio ranges preferably from 0.2:1 to 0.4:1 and preferably from 0.25:1 to 0.35:1 and more particularly equals 0.3:1. The weight-average molecular weight (Mw) of the silicone ranges preferably from 2,000 to 1,000,000, more particularly from 3,500 to 200,000.

Another group of amino silicones corresponding to this definition is represented by the following formula:

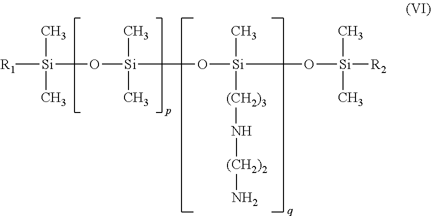

in which:
p and q are numbers such that the sum (p+q) ranges from 1 to 1000, particularly from 50 to 350, and more particularly from 150 to 250; it being possible for p to denote a number from 0 to 999 and in particular from 49 to 349, and more particularly from 159 to 239 and for q to denote a number from 1 to 1000, in particular from 1 to 10, and more particularly from 1 to 5;
R¹, and R², which may be the same or different, represent a hydroxy or C₁-C₄ alkoxy radical, where at least one of the radicals R¹ or R² denotes an alkoxy radical.

The alkoxy radical is preferably a methoxy radical. The hydroxy/alkoxy mole ratio ranges generally from 1:0.8 to 1:1.1 and preferably from 1:0.9 to 1:1 and more particularly equals 1:0.95.

Another group of amino silicones is represented by the following formula:

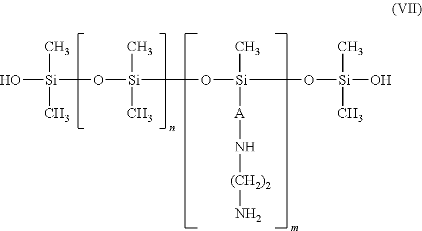

in which:
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;

A denotes a linear or branched alkylene radical containing from 4 to 8 carbon atoms and preferably 4 carbon atoms. This radical is preferably linear.

The weight-average molecular weight (Mw) of these amino silicones ranges preferably from 2000 to 1 000 000 and even more particularly from 3500 to 200 000.

Another group of amino silicones is represented by the following formula:

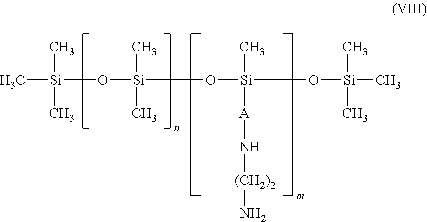

in which:
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;

A denotes a linear or branched alkylene radical containing from 4 to 8 carbon atoms and preferably 4 carbon atoms. This radical is preferably branched.

The weight-average molecular weight (Mw) of these amino silicones ranges preferably from 500 to 1 000 000 and even more particularly from 1000 to 200 000.

Another group of amino silicones is represented by the following formula:

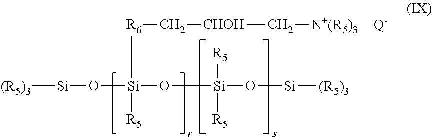

in which:
R₅ represents a monovalent hydrocarbon-based radical containing from 1 to 18 carbon atoms, and in particular a C₁-C₁₈ alkyl or C₂-C₁₈ alkenyl radical, for example methyl;
   R₆ represents a divalent hydrocarbon-based radical, in particular a C₁-C₁₈ alkylene radical or a divalent C₁-C₁₈, for example C₁-C₈, alkylenoxy radical linked to the Si via a SiC bond;
Q- is an anion such as a halide ion, in particular chloride, or an organic acid salt (for example acetate);
r represents a mean statistical value from 2 to 20, and in particular from 2 to 8;
s represents a mean statistical value from 20 to 200, and in particular from 20 to 50.

A group of quaternary ammonium silicones is represented by the following formula:

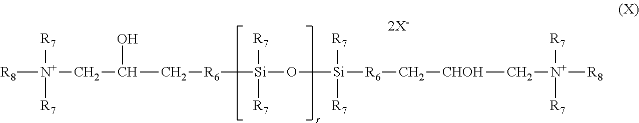

in which:
R₇, which may be identical or different, represent a monovalent hydrocarbon-based radical containing from 1 to 18 carbon atoms, and in particular a C₁-C₁₈ alkyl radical, a C₂-C₁₈ alkenyl radical or a ring containing 5 or 6 carbon atoms, for example methyl;
R₆ represents a divalent hydrocarbon-based radical, in particular a C₁-C₁₈ alkylene radical or a divalent C₁-C₁₈, for example C₁-C₈, alkylenoxy radical linked to the Si via an SiC bond;
R₈, which may be identical or different, represent a hydrogen atom, a monovalent hydrocarbon-based radical containing from 1 to 18 carbon atoms, and in particular a C₁-C₁₈ alkyl radical, a C₂-C₁₈ alkenyl radical or a —R₆—NHCOR₇ radical;
X— is an anion such as a halide ion, in particular chloride, or an organic acid salt (for example acetate);
r represents a mean statistical value from 2 to 200 and in particular from 5 to 100. These silicones are described, for example, in patent application EP-A 0530974.

A group of quaternary ammonium silicones is represented by the following formula:

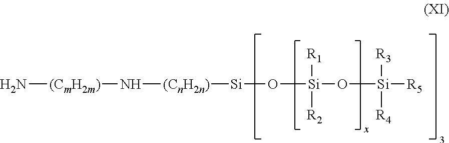

in which:
R₁, R₂, R₃ and R₄, which may be identical or different, denote a C₁-C₄ alkyl radical or a phenyl group;
R₅ denotes a C₁-C₄ alkyl radical or a hydroxyl group;
n is an integer ranging from 1 to 5;
m is an integer ranging from 1 to 5;
and in which x is chosen such that the amine number is between 0.01 and 1 meq/g;
multiblock polyoxyalkylenated amino silicones, of type (AB)n, A being a polysiloxane block and B being a polyoxyalkylenated block containing at least one amine group.

Said silicones are preferably constituted of repeating units having the following general formulae:

[—(SiMe₂O)xSiMe₂-R—N(R″)—R′—O(C₂H₄O)ₐ(C₃H₆O)b—R′—N(H)—R—]

or alternatively

[—(SiMe₂O)xSiMe₂-R—N(R″)—R′—O(C₂H₄O)ₐ(C₃H₆O)b-]
in which:
a is an integer greater than or equal to 1, preferably ranging from 5 to 200, more particularly ranging from 10 to 100;
b is an integer comprised between 0 and 200, preferably ranging from 4 to 100, more particularly between from 5 and 30;
x is an integer ranging from 1 to 10 000, more particularly from 10 to 5000;
R″ is a hydrogen atom or a methyl;
R, which may be identical or different, represent a divalent linear or branched C₂-C₁₂ hydrocarbon-based radical, optionally including one or more heteroatoms such as oxygen; preferably, R denotes an ethylene radical, a linear or branched propylene radical, a linear or branched butylene radical, or a —CH₂CH₂CH₂OCH(OH)CH₂— radical; preferentially R denotes a —CH₂CH₂CH₂OCH(OH)CH₂— radical;
R′, which may be identical or different, represent a divalent linear or branched C₂-C₁₂ hydrocarbon-based radical, optionally including one or more heteroatoms such as oxygen; preferably, R′ denotes an ethylene radical, a linear or branched propylene radical, a linear or branched butylene radical, or a —CH₂CH₂CH₂OCH(OH)CH₂— radical; preferentially R′ denotes —CH(CH₃)—CH₂—.

The siloxane blocks preferably represent between 50 and 95 mol % of the total weight of the silicone, more particularly from 70 to 85 mol %.

The amine content is preferably between 0.02 and 0.5 meq/g of copolymer in a 30% solution in dipropylene glycol, more particularly between 0.05 and 0.2. The weight-average molecular weight (Mw) of the silicone oil is preferably comprised between 5000 and 1,000,000, more particularly between 10,000 and 200,000.

Non-limiting examples of amino-functionalized silicones include bis-hydroxy/methoxy amodimethicones, bis-cetearyl amodimethicone, amodimethicone, bis(C13-15 alkoxy) PG amodimethicones, aminopropyl phenyl trimethicones, aminopropyl dimethicones, bis-amino PEG/PPG-41/3 aminoethyl PG-propyl dimethicones, caprylyl methicones, and a combination thereof. In some instances, a particularly useful amino-functionalized silicone is bis-hydroxy/methoxy amodimethicone, wherein X is isobutyl and one of the R is OH and the other is OCH₃ in the above structure, also known as “Bis-Hydroxy/Methoxy Amodimethicone” and “3-[(2-aminoethyl)amino]-2-methylpropyl Me, di-Me, [(hydroxydimethylsilyl)oxy]- and [(methoxydimethylsilyl)oxy]-terminated.” Bis-hydroxy/methoxy amodimethicone is commercially available under the tradename, “DOWSIL AP-8087 FLUID” from The Dow Chemical Company. A particularly preferred amino-functionalized silicone is amodimethicone.” A non-limiting example of amodimethicone products containing amino silicones having structure (D) re sold by Wacker under the tradename, “BELSIL ADM 652”, “BELSIL ADM 4000 E”, or “BELSIL ADM LOG 1”. A product containing amino silicones having structure (E) is sold by Wacker under the tradename, “FLUID WR 1300.” Additionally or in the alternative, the weight-average molecular weight (Mw) of the silicone ranges preferably from 2,000 to 200,000, even more particularly 5,000 to 100,000, and more particularly from 10,000 to 50,000.

In a preferred embodiment, the one or more amino-functionalized silicones are selected from amodimethicone, bis-hydroxy/methoxy amodimethicone, bis-cetearyl amodimethicone, bis(C13-15 alkoxy) PG amodimethicone, aminopropyl phenyl trimethicone, aminopropyl dimethicone, bis-amino PEG/PPG-41/3 aminoethyl PG-propyl dimethicone, or a combination thereof. In a further preferred embodiment, the amino-functionalized silicone is amodimethicone.

The total amount of the one or more amino-functionalized silicones in the hair treatment compositions will vary. Nonetheless, in various embodiments, the hair treatment compositions include about 0.1 to about 10 wt. % of the one or more amino-functionalized silicones, based on the total weight of the hair treatment composition. In further embodiments, the hair treatment compositions include about 0.1 to about 8 wt. %, about 0.1 to about 5 wt. %, about 0.1 to about 3 wt. %, about 0.1 to about 2 wt. %, about 0.1 to about 1 wt. %, about 0.5 to about 10 wt. %, about 0.5 to about 8 wt. %, about 0.5 to about 5 wt. %, about 0.5 to about 3 wt. %, about 0.5 to about 2 wt. %, or about 0.5 to about 1 wt. %, based on a total weight of the hair treatment composition.

### (g) Water

The amount of water in the hair treatment compositions will vary. Nonetheless, in various embodiments, the hair treatment composition includes about 60 to about 90 wt. % water. In further embodiments, the hair treatment composition includes about 60 to about 85 wt. %, about 60 to about 80 wt. %, about 65 to about 90 wt. %, about 65 to about 85 wt. %, about 65 to about 80 wt. %, about 70 to about 90 wt. %, about 70 to about 85 wt. %, about 70 to about 80 wt. %, about 75 to about 90 wt. %, about 75 to about 85 wt. %, about 75 to about 80 wt. % water, based on a total weight of the hair treatment composition.

### (h) Water-Soluble Solvent

The term “water soluble solvent” is interchangeable with the terms “water soluble organic solvent” and “water-miscible solvent” and means a compound that is liquid at 25° C. and at atmospheric pressure (760 mmHg) and has a solubility of at least 50% in water under these conditions. In some cases, the water-soluble solvent has a solubility of at least 60%, 70%, 80%, or 90%. Non-limiting examples of water-soluble solvents include, for example, organic solvents selected from glycerin, alcohols (for example C₂₋₈, monoalcohols), polyols (polyhydric alcohols), glycols, and a combination thereof.

Non-limiting examples of water-soluble organic solvents include, for example, organic solvents selected from glycerin, alcohols (for example, C₁₋₁₀, C₁₋₈, or C₁₋₄ alcohols), polyols (polyhydric alcohols), glycols, and a combination thereof. Nonlimiting examples of monoalcohols and polyols include ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin.

Further non-limiting examples of water soluble organic solvents include alkanediols (polyhydric alcohols) such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a combination thereof.

Polyhydric alcohols are useful. Examples of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a combination thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a combination thereof.

In a preferred embodiment, the hair treatment composition includes one or more glycols selected from glycerin, propylene glycol, butylene glycol, pentylene glycol, hexylene glycol, caprylyl glycol, dipropylene glycol, a C₂-C₆ monoalcohol (such as ethanol or isopropanol), and combinations thereof.

The total amount of the one or more water-soluble solvents in the hair treatment composition will vary. Nonetheless, in various embodiments, the hair treatment composition includes about 0.1 to about 20 wt. % of the one or more water soluble solvents, based on the total weight of the hair treatment compositions. In further embodiments, the hair treatment composition includes about 0.1 to about 15 wt. %, about 0.1 to about 12 wt. % about 0.1 to about 10 wt. %, about 0.5 to about 20 wt. %, about 0.5 to about 15 wt. %, about 0.5 to about 12 wt. %, about 0.5 to about 10 wt. %, about 1 to about 20 wt. %, about 1 to about 15 wt. %, about 1 to about 12 wt. %, about 1 to about 10 wt. %, about 2 to about 20 wt. %, about 2 to about 15 wt. %, about 2 to about 12 wt. %, about 2 to about 10 wt. %, about 3 to about 20 wt. %, about 3 to about 15 wt. %, about 3 to about 12 wt. %, about 3 to about 10 wt. %, about 5 to about 20 wt. %, about 5 to about 15 wt. %, about 5 to about 12 wt. %, or about 5 to about 10 wt. % of the one or more water-soluble solvents, based on a total weight of the hair treatment composition.

### (i) Thickening Polymers

In certain embodiments, the hair treatment compositions of the instant disclosure include one or more thickening polymers. However, in other embodiments, the hair treatment compositions do not include thickening polymers, i.e., the composition is free or essentially free from thickening polymers. When included, in various embodiments, the one or more thickening polymers may be selected from nonionic thickening polymers, cationic thickening polymers, and a combination thereof.

Nonlimiting examples of nonionic thickening polymers, polyacrylate polymers, polyacrylamide polymers, polysaccharides, gums (e.g. guar gums), and a combination thereof. The cationic thickening polymers include cationic polymers having quaternary amine group or a quaternary ammonium group. In a preferred embodiment, the composition includes one or more cationic thickening polymers selected from polyquaternium-10, polyquaternium-67, and a combination thereof, preferably polyquaternium-67.

The total amount of the one or more thickening polymers, if present, will vary. Nonetheless, in various embodiments, the total amount of the one or more thickening polymers is from about 0.01 to about 5 wt. %, based on the total weight of the composition. In further embodiments, the total amount of the one or more thickening polymers is from about 0.01 to about 4 wt. %, about 0.01 to about 3 wt. %, about 0.01 to about 2 wt. %, about 0.01 to about 1 wt. %, about 0.05 to about 5 wt. %, about 0.05 to about 4 wt. %, about 0.05 to about 3 wt. %, about 0.05 to about 2 wt. %, about 0.05 to about 1 wt. %, about 0.1 to about 5 wt. %, about 0.1 to about 4 wt. %, about 0.1 to about 3 wt. %, about 0.1 to about 2 wt. %, or about 0.1 to about 1 wt. %, based on the total weight of the composition.

i. Nonionic Thickening Polymers

In various embodiments, the compositions of the present disclosure include one or more nonionic thickening polymers. Nonlimiting examples of nonionic thickening polymers include methyl hydroxypropyl cellulose, Gellan Gum (“Kelcogel” from CP Kelco), polysaccharide, gum, hydroxyl propyl cellulose “(Methocel” from Dow/Amerchol), hydroxyl propyl methyl cellulose (“Klucel” from Hercules), hydroxyl ethyl cellulose, polyalkylene glycols, and combinations thereof. Particularly useful nonionic polymers include polysaccharide gum, hydroxyl propyl cellulose, hydroxyl propyl methyl cellulose, or combinations thereof.

The one or more nonionic thickening polymers may include polysaccharides, especially polysaccharides selected from modified or unmodified starches (such as those derived, for example, from cereals, for instance wheat, corn or rice, from vegetables, for instance yellow pea, and tubers, for instance potato or cassava), amylose, amylopectin, glycogen, dextrans, celluloses and derivatives thereof (methylcellulose loses, hydroxyalkylcelluloses, ethylhydroxyethylcelluloses), xylans including glucuronoxylans and arabinoxylans, glucans including xyloglucans, arabans, galactans including arabinogalactans, chitin, agars, locust bean gums, mannans including glucomannans and galactomannans such as guar gums and nonionic derivatives thereof (hydroxypropyl guar), and combinations thereof.

In a preferred embodiment, the one or more nonionic thickening agents are selected from polyacrylate polymers, polyacrylamide polymers, polysaccharides, gums, and a combination thereof, more preferably selected from polysaccharides, gums, and combinations thereof, and even more preferably selected from guar gums, modified guar gums such as hydroxypropyl guar, and celluloses, for example, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and combinations thereof. In some instances, the cellulose is selected from water soluble cellulose derivatives (for example, methyl cellulose, methylhydroxypropyl cellulose, hydroxyethyl cellulose, hydroxypropyl cellulose, cellulose sulfate sodium salt), and combinations thereof. Furthermore, in some instance, the cellulose is preferably hydroxypropylcellulose (HPC).

The total amount of the one or more nonionic thickening polymers, if present, will vary. Nonetheless, in various embodiments, the total amount of the one or more nonionic thickening polymers is from about 0.01 to about 5 wt. %, based on the total weight of the composition. In further embodiments, the total amount of the one or more thickening polymers is from about 0.01 to about 4 wt. %, about 0.01 to about 3 wt. %, about 0.01 to about 2 wt. %, about 0.01 to about 1 wt. %, about 0.05 to about 5 wt. %, about 0.05 to about 4 wt. %, about 0.05 to about 3 wt. %, about 0.05 to about 2 wt. %, about 0.05 to about 1 wt. %, about 0.1 to about 5 wt. %, about 0.1 to about 4 wt. %, about 0.1 to about 3 wt. %, about 0.1 to about 2 wt. %, or about 0.1 to about 1 wt. %, based on the total weight of the composition.

ii. Cationic Thickening Polymers

In various embodiments, suitable cationic thickening polymers for use in the compositions of the present invention contain cationic nitrogen-containing moieties such as quaternary ammonium or cationic protonated amino moieties. The cationic protonated amines can be primary, secondary, or tertiary amines (preferably secondary or tertiary), depending upon the particular species. Anionic counterions can be used in association with the cationic thickening polymers. Non limiting examples of such counterions include halides (e.g., chloride, fluoride, bromide, iodide), sulfate, and methylsulfate. Non limiting examples of cationic thickening polymers are described in the CTFA Cosmetic Ingredient Dictionary, 3rd edition, edited by Estrin, Crosley, and Haynes, (The Cosmetic, Toiletry, and Fragrance Association, Inc., Washington, D.C. (1982)), which is incorporated herein by reference in its entirety. More specifically, in various embodiments, the one or more cationic thickening polymer be selected from copolymers of vinyl monomers having cationic protonated amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, or vinyl pyrrolidone.

Nonlimiting examples of cationic protonated amino and quaternary ammonium monomers, for inclusion in the cationic thickening polymers, include vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts.

Additional nonlimiting examples of useful cationic thickening polymers include copolymers of 1-vinyl-2-pyrrolidone and 1-vinyl-3-methylimidazolium salt (e.g., chloride salt) (referred to in the industry by the Cosmetic, Toiletry, and Fragrance Association, “CTFA”, as Polyquaternium-16); copolymers of 1-vinyl-2-pyrrolidone and dimethylaminoethyl methacrylate (referred to in the industry by CTFA as Polyquaternium-11); cationic diallyl quaternary ammonium-containing polymers, including, for example, dimethyldiallylammonium chloride homopolymer, copolymers of acrylamide and dimethyldiallylammonium chloride (referred to in the industry by CTFA as Polyquaternium 6 and Polyquaternium 7, respectively); amphoteric copolymers of acrylic acid including copolymers of acrylic acid and dimethyldiallylammonium chloride (referred to in the industry by CTFA as Polyquaternium 22), terpolymers of acrylic acid with dimethyldiallylammonium chloride and acrylamide (referred to in the industry by CTFA as Polyquaternium 39), and terpolymers of acrylic acid with methacrylamidopropyl trimethylammonium chloride and methylacrylate (referred to in the industry by CTFA as Polyquaternium 47). Preferred cationic substituted monomers are the cationic substituted dialkylaminoalkyl acrylamides, dialkylaminoalkyl methacrylamides, and combinations thereof.

In addition to the above, in certain embodiments, the one or more cationic thickening polymers may be selected from cationic polysaccharide polymers, such as cationic cellulose derivatives and cationic starch derivatives. Preferred cationic cellulose polymers are salts of hydroxyethyl cellulose reacted with trimethyl ammonium substituted epoxide, referred to in the industry (CTFA) as Polyquaternium 10 and available from Amerchol Corp. (Edison, N.J., USA) in their “Polymer LR,” “Polymer JR,” and “Polymer KG” series of polymers. Other suitable types of cationic cellulose include the polymeric quaternary ammonium salts of hydroxyethyl cellulose reacted with lauryl dimethyl ammonium-substituted epoxide referred to in the industry (CTFA) as Polyquaternium 24. These materials are available from Amerchol Corp. under the tradename, “Polymer LM-200.” Other suitable cationic thickening polymers include cationic guar gum derivatives, such as guar hydroxypropyltrimonium chloride, specific examples of which include the “Jaguar” series commercially available from Rhone-Poulenc Incorporated and the “N-Hance” series commercially available from Aqualon Division of Hercules, Inc. Other suitable cationic polymers include quaternary nitrogen-containing cellulose ethers.

In a preferred embodiment, the one or more cationic thickening agents are selected from cellulose based cationic polymers, in particular those selected from polyquaternium-10, polyquaternium-24, polyquaternium-27, polyquaternium-67, polyquaternium-72, and a combination thereof. In a particularly preferred embodiment, the compositions of the instant disclosure include polyquaternium-67.

The total amount of the one or more cationic thickening polymers, if present, will vary. Nonetheless, in various embodiments, the total amount of the one or more cationic thickening polymers is from about 0.01 to about 5 wt. %, based on the total weight of the composition. In further embodiments, the total amount of the one or more thickening polymers is from about 0.01 to about 4 wt. %, about 0.01 to about 3 wt. %, about 0.01 to about 2 wt. %, about 0.01 to about 1 wt. %, about 0.05 to about 5 wt. %, about 0.05 to about 4 wt. %, about 0.05 to about 3 wt. %, about 0.05 to about 2 wt. %, about 0.05 to about 1 wt. %, about 0.1 to about 5 wt. %, about 0.1 to about 4 wt. %, about 0.1 to about 3 wt. %, about 0.1 to about 2 wt. %, or about 0.1 to about 1 wt. %, based on the total weight of the composition.

(j) Surfactants Other than the Cationic Surfactants of (b) and (c)

The hair treatment compositions of the instant disclosure optionally include one or more nonionic surfactants, amphoteric surfactants, or a combination thereof. For example, if present, the hair treatment composition may include about 0.01 to about 10 wt. % of one or more nonionic surfactants, one or more amphoteric surfactants, or combination thereof, based on a total weight of the hair treatment composition. In further embodiments, the hair treatment composition includes about 0.01 to about 8 wt. %, about 0.01 to about 5 wt. %, about 0.01 to about 3 wt. %, about 0.01 to about 1 wt. %, about 0.1 to about 10 wt. %, about 0.1 to about 8 wt. %, about 0.1 to about 5 wt. %, about 0.1 to about 3 wt. %, about 0.1 to about 2 wt. %, or about 0.01 to about 1 wt. % one or more nonionic surfactants, one or more amphoteric surfactants, or combination thereof based on a total weight of the hair treatment composition.

### Nonionic Surfactants

Nonlimiting examples of nonionic surfactants include: alkanolamides; alkyl polyglucosides; polyoxyalkylenated nonionic surfactants; polyglycerolated nonionic surfactants; ethoxylated fatty esters; alcohols, alpha-diols, alkylphenols and esters of fatty acids, being ethoxylated, propoxylated or glycerolated; copolymers of ethylene oxide and/or of propylene oxide; condensates of ethylene oxide and/or of propylene oxide with fatty alcohols; polyethoxylated fatty amides; ethoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C₆-C₂₄)alkylpolyglycosides; N—(C₆-C₂₄)alkylglucamine derivatives, amine oxides such as (C₁₀-C₁₄)alkylamine oxides or N—(C₁₀-C₁₄) acylaminopropylmorpholine oxides; and combinations thereof.

In various embodiments, the one or more nonionic surfactants may preferably be chosen from polyoxyalkylenated or polyglycerolated nonionic surfactants. The oxyalkylene units are more particularly oxyethylene or oxypropylene units, or a combination thereof, and are preferably oxyethylene units.

The alkoxylated nonionic surfactants may be chosen from alkoxylated alcohols, alkoxylated fatty alcohols, alkoxylated polyol esters such as polyethylene glycol ethers of fatty alcohols, polyethylene glycol ethers of esters, and polyethylene glycol ethers of glycerides, and combinations thereof. Non-limiting examples of polyethylene glycol ethers of esters include ethoxylated fatty esters. Further discussion of non-limiting examples of the alkoxylated nonionic surfactants are provided below. In some instances, the alkoxylated nonionic surfactants are chosen from PEG-55 propylene glycol oleate, PEG-6 propylene glycol caprylate/caprate, PEG-8 propylene glycol cocoate, PEG-55 propylene glycol oleate, PEG-75 propylene glycol stearate, PEG-25 propylene glycol stearate, PEG-7 glyceryl cocoate, PEG-30 glyceryl cocoate, laureth-2, laureth-3, laureth-4, PEG-200 glyceryl stearate, PEG-120 propylene glycol stearate, PEG-6 Caprylic/Capric Glycerides, and a combination thereof.

“Alkoxylated nonionic surfactant” as used herein means a compound having at least one alkoxylated portion (—(CH2)nO—, where n is an integer from 1 to 300, preferably 2 to 200, or more preferably 2 to 150, even more preferably 2 to 120, or most preferably, 2 to 100).

### Alkoxylated Fatty Alcohol

“Alkoxylated fatty alcohol” as used herein means a compound having at least one fatty portion (e.g. about 8 carbon atoms or more) and at least one alkoxylated portion (—(CH2)nO—, where n is an integer of 1 or more). The alkoxylated fatty alcohols of the present invention preferably have an HLB (hydrophilic-lipophilic balance) value from 1-20, including all ranges and subranges therebetween, with HLB values ranging from 1 to 5 (particularly 3 to 5) or from 15-20 (particularly 16 to 18) being preferred. The alkoxylated fatty alcohol may be chosen from ethoxylated fatty alcohols, propoxylated fatty alcohols, and combinations thereof.

The alkoxylated fatty alcohol can be chosen from di-alkyl, tri-alkyl- and combinations of di-alkyl and tri-alkyl substituted ethoxylated polymers. They can also be chosen from mono-alkyl, di-alkyl, tri-alkyl, tetra-alkyl substituted alkyl ethoxylated polymers and all combinations thereof. The alkyl group can be saturated or unsaturated, branched or linear and contain a number of carbon atoms preferably from about 12 carbon atoms to about 50 carbon atoms, including all ranges and subranges therebetween, for example, 20 to 40 carbon atoms, 22 to 24 carbon atoms, 30 to 50 carbon atoms, and 40 to 60 carbon atoms. Preferably, the fatty portion contains a mixture of compounds of varying carbon atoms such as, for example, C₂₀-C₄₀ compounds, C₂₂-C₂₄ compounds, C₃₀-C₅₀ compounds, and C₄₀-C₆₀ compounds.

Preferably, the alkoxylated portion of the alkoxylated fatty alcohols of the present disclosure contain 2 or more alkoxylation units, preferably from 2 to 20 alkoxylation units, preferably from 2 to 12 alkoxylation units, preferably from 10 to 200 alkoxylation units, preferably from 20 to 150 alkoxylation units, and preferably from 25 to 100 alkoxylation units, including all ranges and subranges therebetween. Also preferably, the alkoxylation units contain 2 carbon atoms (ethoxylation units) and/or 3 carbon atoms (propoxylation units).

The amount of alkoxylation can also be determined by the percent by weight of the alkoxylated portion with respect to the total weight of the compound. Suitable weight percentages of the alkoxylated portion with respect to the total weight of the compound include, but are not limited to, 10 percent to 95 percent, preferably 20 percent to 90 percent, including all ranges and subranges therebetween with 75 percent to 90 percent (particularly 80 percent to 90 percent) or 20 percent to 50 percent being preferred.

Preferably, the alkoxylated fatty alcohols of the present invention have a number average molecular weight (Mn) greater than 500, preferably from 500 to 5,000, including all ranges and subranges therebetween such as, for example, Mn of 500 to 1250 or an Mn of 2,000 to 5,000.

Suitable examples of alkoxylated fatty alcohols include: laureth-3, laureth-4, laureth-7, laureth-9, laureth-12, laureth-23, ceteth-10, steareth-10, steareth-6, steareth-2, steareth-100, beheneth-5, beheneth-5, beheneth-10, oleth-10, Pareth alcohols, trideceth-3, trideceth-10, trideceth-12, C12-13 pareth-3, C12-13 pareth-23, C11-15 pareth-7, PEG hydrogenated castor oil, PEG-75 lanolin, polysorbate-80, polysobate-20, PPG-5 ceteth-20, PEG-55 Propylene Glycol Oleate, glycereth-26 (PEG-26 Glyceryl Ether), PEG 120 methyl glucose dioleate, PEG 120 methyl glucose trioleate, PEG 150 pentaerythrityl tetrastearate, and combinations thereof.

### Alkoxylated Polyol Esters

The alkoxylated polyol esters may be chosen from pegylated derivatives of propylene glycol oleate, propylene glycol caprylate/caprate, propylene glycol cocoate, propylene glycol stearate, and a combination thereof. In certain embodiments, the alkoxylated polyol esters are chosen from PEG-55 propylene glycol oleate, PEG-6 propylene glycol caprylate/caprate, PEG-8 propylene glycol cocoate, PEG-25 propylene glycol stearate, and PEG-120 propylene glycol stearate, and a combination thereof. In some instances, the polyol ester is or includes PEG-55 propylene glycol oleate. While the alkoxylated polyol esters comprise PEG-200 glyceryl stearate in some embodiments, in other embodiments PEG-200 glyceryl stearate may be excluded. Additionally and/or alternatively, the polyol esters may be chosen from ethoxylated fatty acid esters of sorbitan comprising from 2 to 30 mol of ethylene oxide.

In some cases, the polyol ester may be selected from esters of polyols with fatty acids with a saturated or unsaturated chain containing for example from 8 to 24 carbon atoms, preferably 12 to 22 carbon atoms, and alkoxylated derivatives thereof, preferably with a number of alkyleneoxide of from 10 to 200, and more preferably from 10 to 100, such as glyceryl esters of a C₈-C₂₄, preferably C₁₂-C₂₂, fatty acid or acids and alkoxylated derivatives thereof, preferably with a number of alkyleneoxide of from 10 to 200, and more preferably from 10 to 100; polyethylene glycol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sorbitol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sugar (sucrose, glucose, alkylglycose) esters of a C₈-C₂₄, preferably C₁₂-C₂₂, fatty acid or acids and alkoxylated derivatives thereof, preferably with a number of alkyleneoxide of from 10 to 200, and more preferably from 10 to 100; ethers of fatty alcohols; ethers of sugar and a C₈-C₂₄, preferably C₁₂-C₂₂, fatty alcohol or alcohols; and combinations thereof.

Examples of ethoxylated fatty esters that may be mentioned include the adducts of ethylene oxide with esters of lauric acid, palmitic acid, stearic acid or behenic acid, and combinations thereof, especially those containing from 9 to 100 oxyethylene groups, such as PEG-9 to PEG-50 laurate (as the INCI names: PEG-9 laurate to PEG-50 laurate); PEG-9 to PEG-50 palmitate (as the INCI names: PEG-9 palmitate to PEG-50 palmitate); PEG-9 to PEG-50 stearate (as the INCI names: PEG-9 stearate to PEG-50 stearate); PEG-9 to PEG-50 palmitostearate; PEG-9 to PEG-50 behenate (as the INCI names: PEG-9 behenate to PEG-50 behenate); polyethylene glycol 100 EO monostearate (INCI name: PEG-100 stearate); and combinations thereof.

Sources of unsaturated polyol esters of glycerol include synthesized oils, natural oils (e.g., vegetable oils, algae oils, bacterial derived oils, and animal fats), combinations of these, and the like. Non-limiting examples of vegetable oils include Abyssinian oil, Almond oil, Apricot oil, Apricot Kernel oil, Argan oil, Avocado oil, Babassu oil, Baobab oil, Black Cumin oil, Black Currant oil, Borage oil, Camelina oil, Carinata oil, Canola oil, Castor oil, Cherry Kernel oil, Coconut oil, Corn oil, Cottonseed oil, Echium oil, Evening Primrose oil, Flax Seed oil, Grape Seed oil, Grapefruit Seed oil, Hazelnut oil, Hemp Seed oil, Jatropha oil, Jojoba oil, Kukui Nut oil, Linseed oil, Macadamia Nut oil, Meadowfoam Seed oil, Moringa oil, Neem oil, Olive oil, Palm oil, Palm Kernel oil, Peach Kernel oil, Peanut oil, Pecan oil, Pennycress oil, Perilla Seed oil, Pistachio oil, Pomegranate Seed oil, Pongamia oil, Pumpkin Seed oil, Raspberry oil, Red Palm Olein, Rice Bran oil, Rosehip oil, Safflower oil, Seabuckthorn Fruit oil, Sesame Seed oil, Shea Olein, Sunflower oil, Soybean oil, Tonka Bean oil, Tung oil, Walnut oil, Wheat Germ oil, High Oleoyl Soybean oil, High Oleoyl Sunflower oil, High Oleoyl Safflower oil, High Erucic Acid Rapeseed oil, combinations of these, and the like. Non-limiting examples of animal fats include lard, tallow, chicken fat, yellow grease, fish oil, emu oil, combinations of these, and the like. Non-limiting example of a synthesized oil includes tall oil, which is a byproduct of wood pulp manufacture. In some embodiments, the natural oil is refined, bleached, and/or deodorized.

The polyol esters may optionally be a natural polyol esters chosen from vegetable oil, an animal fat, an algae oil and combinations thereof; and said synthetic polyol ester is derived from a material selected from the group consisting of ethylene glycol, propylene glycol, glycerol, polyglycerol, polyethylene glycol, polypropylene glycol, poly(tetramethylene ether) glycol, pentaerythritol, dipentaerythritol, tripentaerythritol, trimethylolpropane, neopentyl glycol, a sugar, in one aspect, sucrose, and combinations thereof.

Additional non-limiting examples of nonionic surfactants that may optionally be used in the cleansing composition include and/or may be chosen from alkanolamides; polyoxyalkylenated nonionic surfactants; polyglycerolated nonionic surfactants; ethoxylated fatty esters; alcohols, alpha-diols, alkylphenols and esters of fatty acids, being ethoxylated, propoxylated or glycerolated; copolymers of ethylene oxide and/or of propylene oxide; condensates of ethylene oxide and/or of propylene oxide with fatty alcohols; polyethoxylated fatty amides; ethoxylated oils from plant origin; fatty acid esters of sucrose; fatty acid esters of polyethylene glycol; N—(C₆-C₂₄)alkylglucamine derivatives, amine oxides such as (C₁₀-C₁₄)alkylamine oxides or N—(C₁₀-C₁₄) acylaminopropylmorpholine oxides; and combinations thereof.

### Alkoxylated Glycerides

Non-limiting examples of alkoxylated glycerides that may be suitable in certain embodiments include PEG-6 almond glycerides, PEG-20 almond glycerides, PEG-35 almond glycerides, PEG-60 almond glycerides, PEG-192 apricot kernel glycerides, PEG-11 avocado glycerides, PEG-14 avocado glycerides, PEG-11 babassu glycerides, PEG-42 babassu glycerides, PEG-4 caprylic/capric glycerides, PEG-6 caprylic/capric glycerides, PEG-7 caprylic/capric glycerides, PEG-8 caprylic/capric glycerides, PEG-11 cocoa butter glycerides, PEG-75 cocoa butter glycerides, PEG-7 cocoglycerides, PEG-9 cocoglycerides, PEG-20 corn glycerides, PEG-60 corn glycerides, PEG-20 evening primrose glycerides, PEG-60 evening primrose glycerides, PEG-5 hydrogenated corn glycerides, PEG-8 hydrogenated fish glycerides, PEG-20 hydrogenated palm glycerides, PEG-6 hydrogenated palm/palm kernel glyceride, PEG-16 macadamia glycerides, PEG-70 mango glycerides, PEG-13 mink glycerides, PEG-25 moringa glycerides, PEG-42 mushroom glycerides, PEG-2 olive glycerides, PEG-6 olive glycerides, PEG-7 olive glycerides, PEG-10 olive glycerides, PEG-40 olive glycerides, PEG-18 palm glycerides, PEG-12 palm kernel glycerides, PEG-45 palm kernel glycerides, PEG-60 Passiflora edulis seed glycerides, PEG-60 Passiflora incarnata seed glycerides, PEG-45 safflower glycerides, PEG-60 shea butter glycerides, PEG-75 shea butter glycerides, PEG-75 shorea butter glycerides, PEG-35 soy glycerides, PEG-75 soy glycerides, PEG-2 sunflower glycerides, PEG-7 sunflower glycerides, PEG-10 sunflower glycerides, PEG-13 sunflower glycerides, PEG-5 tsubakiate glycerides, PEG-10 tsubakiate glycerides, PEG-20 tsubakiate glycerides, PEG-60 tsubakiate glycerides, and sodium PEG-8 palm glycerides carboxylate.

In some embodiments, the at least one alkoxylated nonionic surfactant includes alkoxylated polyol esters such as polyethylene glycol ethers of esters. For example, the polyethylene glycol ethers of esters may be chosen from PEG-55 propylene glycol oleate, PEG-6 propylene glycol caprylate/caprate, PEG-8 propylene glycol cocoate, PEG-25 propylene glycol stearate, PEG-7 glyceryl cocoate, PEG-30 glyceryl cocoate, laureth-2, laureth-3, laureth-4, PEG-200 glyceryl stearate, PEG-55 propylene glycol oleate. In further embodiments, the alkoxylated nonionic surfactants comprise a polyethylene glycol ethers of esters and at least one alkoxylated nonionic surfactant other than a polyethylene glycol ether of an ester.

In an embodiment, the at least one alkoxylated nonionic surfactant comprises at least one polyethylene glycol ether of fatty alcohols. For example, the polyethylene glycol ether of fatty alcohol may be chosen from laureth-2, laureth-3, laureth-4, steareth-20, or combinations thereof. The polyethylene glycol ether of fatty alcohols may have from 8 to 30 carbon atoms and in particular from 10 to 22 carbon atoms, such as polyethylene glycol ethers of cetyl alcohol, of stearyl alcohol or of cetearyl alcohol (mixture of cetyl alcohol and stearyl alcohol). Mention may be made, for example, of ethers including from 1 to 200 and preferably from 2 to 100 oxyethylene groups, such as those with the CTFA name Ceteareth-20 or Ceteareth-30, and combinations thereof.

In an embodiment, the at least one alkoxylated nonionic surfactant comprises at least one polyethylene glycol ether of glycerides. For example, the polyethylene glycol ether of glyceride may be chosen from PEG-6 Caprylic/Capric Glycerides). In another embodiment, the cleansing composition comprises at least two alkoxylated nonionic surfactant. Preferably, one of the at least two alkoxylated nonionic surfactants is PEG-55 propylene glycol oleate.

### Glucosides

In some embodiments, the one or more glucosides include those chosen from lauryl glucoside, octyl glucoside, decyl glucoside, coco glucoside, caprylyl/capryl glucoside, sodium lauryl glucose carboxylate, and a combination thereof. Additionally or alternatively, the glucosides may be an alkyl polyglucoside that is chosen from glycerol (C₆-C₂₄)alkylpolyglycosides including, e.g., polyethoxylated fatty acid mono or diesters of glycerol (C₆-C₂₄)alkylpolyglycosides. Additional alkyl polyglucosides that may be suitably incorporated, in some instances, in the cleansing composition includes alkyl polyglucosides having a structure according to the following formula (XII):

R¹—O—(R²O)ₙ—(x) (XII)
wherein R¹ is an alkyl group having 8-18 carbon atoms;
R² is an ethylene or propylene group;
Z is a saccharide group with 5 to 6 carbon atoms;
n is an integer from 0 to 10; and
x is an integer from 1 to 5.

Alkyl poly glucosides may, in some instances, include lauryl glucoside, octyl glucoside, decyl glucoside, coco glucoside, caprylyl/capryl glucoside, and sodium lauryl glucose carboxylate. Typically, the at least one alkyl poly glucoside compound is selected from the group consisting of lauryl glucoside, decyl glucoside and coco glucoside. In some instances, decyl glucoside is particularly preferred.

### Alkanolamides

Non-limiting examples of alkanolamides include fatty acid alkanolamides. The fatty acid alkanolamides may be fatty acid monoalkanolamides or fatty acid dialkanolamides or fatty acid isoalkanolamides, and may have a C₂₋₈ hydroxyalkyl group (the C₂₋₈ chain can be substituted with one or more than one-OH group). Non-limiting examples include fatty acid diethanolamides (DEA) or fatty acid monoethanolamides (MEA), fatty acid monoisopropanolamides (MIPA), fatty acid diisopropanolamides (DIPA), and fatty acid glucamides (acyl glucamides).

Suitable fatty acid alkanolamides may include those formed by reacting an alkanolamine and a C₆-C₃₆ fatty acid. Examples include, but are not limited to: oleic acid diethanolamide, myristic acid monoethanolamide, soya fatty acids diethanolamide, stearic acid ethanolamide, oleic acid monoisopropanolamide, linoleic acid diethanolamide, stearic acid monoethanolamide (Stearamide MEA), behenic acid monoethanolamide, isostearic acid monoisopropanolamide (isostearamide MIPA), erucic acid diethanolamide, ricinoleic acid monoethanolamide, coconut fatty acid monoisopropanolamide (cocoamide MIPA), coconut acid monoethanolamide (Cocamide MEA), palm kernel fatty acid diethanolamide, coconut fatty acid diethanolamide, lauric diethanolamide, polyoxyethylene coconut fatty acid monoethanolamide, coconut fatty acid monoethanolamide, lauric monoethanolamide, lauric acid monoisopropanolamide (lauramide MIPA), myristic acid monoisopropanolamide (Myristamide MIPA), coconut fatty acid diisopropanolamide (cocamide DIPA), and combinations thereof.

In some instances, the fatty acid alkanolamides preferably include cocamide MIPA, cocamide DEA, cocamide MEA, cocamide DIPA, and combinations thereof. In particular, the fatty acid alkanolamide may be cocamide MIPA, which is commercially available under the tradename, “EMPILAN” from Innospec Active Chemicals.

Fatty acid alkanolamides include those of the following formula (XIII):

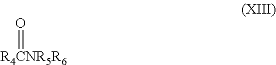

wherein R₄ is an alkyl chain of 4 to 20 carbon atoms (R₄ may be, for example, selected from lauric acid, coconut acid, palmitic acid, myristic acid, behenic acid, babassu fatty acid, isostearic acid, stearic acid, corn fatty acid, soy fatty acid, shea butter fatty acids, caprylic acid, capric acid, and combinations thereof); wherein R₅ is selected from —CH₂OH, —CH₂CH₂OH, —CH₂CH₂CH₂OH, —CH₂(CHOH)₄CH₂OH, -benzyl, and combinations thereof; and wherein R₆ is selected from —H, —CH₃, —CH₂OH, —CH₂CH₃, —CH₂CH₂OH, —CH₂CH₂CH₂OH, —CH₂(CHOH)₄CH₂OH, -benzyl, and combinations thereof.

In some instances, the one or more of the fatty acid alkanolamides include one or more acyl glucamides, e.g., acyl glucamides having a carbon chain length of 8 to 20. Non-limiting examples include lauroyl/myristoyl methyl glucamide, capryloyl/capryl methyl glucamide, lauroyl methyl glucamide, myristoyl methyl glucamide, capryloyl methyl glucamide, capryl methyl glucamide, cocoyl methyl glucamide, capryloyl/caproyl methyl glucamide, cocoyl methyl glucamide, lauryl methylglucamide, oleoyl methylglucamide oleate, stearoyl methylglucamide stearate, sunfloweroyl methylglucamide, and tocopheryl succinate methylglucamide.

### Sorbitan Derivatives

Suitable sorbitan derivatives that may be incorporated into the plurality of nonionic surfactants include those chosen from polysorbate-20 (POE(20) sorbitan monolaurate), polysorbate-21 (POE(4) sorbitan monolaurate), polysorbate-40 (POE(20) sorbitan monopalmitate), polysorbate-60 (POE(20) sorbitan monostearate), polysorbate-61 (POE(4) sorbitan monostearate), polysorbate-65 (POE(20) sorbitan tristearate), polysorbate-80 (POE(20) sorbitan monooleate), polysorbate-81 (POE(4) sorbitan monooleate), polysorbate 85 (POE(20) Sorbitan Trioleate), sorbitan isostearate, sorbitan monolaurate, sorbitan monooleate, sorbitan monopalmitate, sorbitan monostearate, sorbitan sesquioleate, sorbitan trioleate, sorbitan tristearate, and a combination thereof.

Additional and/or alternative sorbitan derivatives include sorbitan esters including, e.g., esters of C₁₆-C₂₂ fatty acid and of sorbitan that were formed by esterification, with sorbitol, of at least one fatty acid comprising at least one saturated or unsaturated linear alkyl chain respectively having from 16 to 22 carbon atoms. These esters can be chosen in particular from sorbitan stearates, behenates, arachidates, palmitates or oleates, and their mixtures. Examples of optional sorbitan esters include sorbitan monostearate (INCI name: Sorbitan stearate) sold by Croda under the tradename, “Span 60,” the sorbitan tristearate sold by Croda under the tradename, “Span 65 V”, the sorbitan monopalmitate (INCI name: Sorbitan palmitate) sold by Croda under the tradename, “Span 40,” the sorbitan monooleate sold by Croda under the tradename, “Span 80 V,” or the sorbitan trioleate sold by Uniqema under the tradename, “Span 85 V”. A preferable sorbitan ester is sorbitan tristearate.

### Silicone Surfactants

As the name indicates, silicone surfactants include silicone. Useful silicone surfactants include PEG/PPG dimethicones, for example, those having from 3 to 30 ethoxy units and from 3 to 30 propoxy units. Nonlimiting examples include PEG/PPG-3/10 dimethicone, PEG/PPG-4/12 dimethicone, PEG/PPG-6/11 dimethicone, PEG/PPG-8/14 dimethicone, PEG/PPG-14/4 dimethicone, PEG/PPG-15/15 dimethicone, PEG/PPG-16/2 dimethicone, PEG/PPG-17/18 dimethicone, PEG/PPG-18/18 dimethicone, PEG/PPG-19/19 dimethicone, PEG/PPG-20/6 dimethicone, PEG/PPG-20/15 dimethicone, PEG/PPG-20/20 Dimethicone, PEG/PPG-20/23 dimethicone, PEG/PPG-20/29 dimethicone, PEG/PPG-22/23 dimethicone, PEG/PPG-22/24 dimethicone, PEG/PPG-23/6 dimethicone, PEG/PPG-25/25 dimethicone, PEG/PPG-27/27, bis-PEG/PPG 18/6 dimethicone, PEG/PPG 30/10 dimethicone, or combinations thereof. In various embodiments, the one or more PEG/PPG-dimethicones are selected from PEG/PPG-3/10 dimethicone, PEG/PPG-4/12 dimethicone, PEG/PPG-6/11 dimethicone, PEG/PPG-8/14 dimethicone, PEG/PPG-14/4 dimethicone, PEG/PPG-15/15 dimethicone, PEG/PPG-16/2 dimethicone, or combinations thereof. A particularly suitable PEG/PPG-dimethicone is PEG/PPG-4/12 dimethicone.

### Amphoteric Surfactants

Nonlimiting examples of amphoteric surfactants include alkyl amphoproprionates, betaines, alkyl sultaines, alkyl amphoacetates, and combinations thereof. In some instances, it is preferable to include one or more alkyl betaines.

### Alkyl Amphopropionates

In some instances, the cleansing compositions preferably include one or more alkyl amphopropionates. Non-limiting examples of alkyl amphopropionates include cocoamphopropionate, cornamphopropionate, caprylamphopropionate, cornamphopropionate, caproamphopropionate, oleoamphopropionate, isostearoamphopropionate, stearoamphopropionate, lauroamphopropionate, salts thereof, and combinations thereof. Sodium cocoamphopropionate is a particularly useful alkyl amphopropionate that can be included in the cleansing compositions.

### Betaines

Particularly useful betaines include, for example, coca betaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, and combinations thereof. Typically, at least one betaine compound is selected from coco betaine, cocamidopropyl betaine, behenyl betaine, capryl/capramidopropyl betaine, and lauryl betaine, and combinations thereof. Particularly preferred betaines include coco betaine and cocamidopropyl betaine.

### Alkyl Sulltaines

Non-limiting examples of alkyl sultaines include hydroxyl sultaines of formula (XIV)

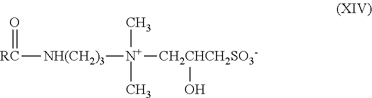

wherein R is an alkyl group having 8-18 carbon atoms. More specific examples include, but are not limited to cocamidopropyl hydroxysultaine, lauryl hydroxysultaine, and a combination thereof.

### (k) Miscellaneous Ingredients

The compositions the instant disclosure may optionally include (or optionally exclude) one more miscellaneous ingredients. Miscellaneous ingredients are ingredients that are compatible with the hair treatment compositions and do not disrupt or materially affect the basic and novel properties of the compositions. Nonlimiting examples of miscellaneous ingredients include preservatives, fragrances, pH adjusters, salts, chelating agents, buffers, antioxidants, flavonoids, vitamins, botanical extracts, UV filtering agents, proteins, protein hydrolysates and/or isolates, fillers (e.g., organic and/or inorganic fillers such as talc, calcium carbonate, silica, etc.) composition colorants, etc.

In various embodiments, the compositions of the instant disclosure include one or more miscellaneous ingredients selected from preservatives, fragrances, pH adjusters, salts, chelating agents, buffers, amino acids, composition colorants, fillers (such as talc, calcium carbonate, silica, including hydrated silica), vitamins, botanical extracts, and a combination thereof. For example, the compositions may include silica (or hydrated silica), tocopherol, fragrances, or a combination thereof.

In the context of the instant disclosure, a “composition colorant” is a compound that colors the composition but does not have an appreciable coloring effect on hair. In other words, the composition colorant is included to provide a color to the composition for aesthetic appeal but is not intended to impart coloring properties to hair. As an example, hair styling gels, can be found in a variety of different colors (e.g., light blue, light pink, etc.) yet application of the styling gel to hair does not visibly change the color of the hair.

The total amount of the one or more miscellaneous ingredients, if present, will vary. Nonetheless, in various embodiments, the compositions of the instant disclosure include from about 0.001 to about 10 wt. % of one or more miscellaneous ingredients, based on the total weight of the composition. In further embodiments, the compositions include from about 0.001 to about 5 wt. %, about 0.001 to about 3 wt. %, about 0.01 to about 10 wt. %, about 0.01 to about 5 wt. %, about 0.01 to about 3 wt. %, about 0.1 to about 10 wt. %, about 0.1 to about 5 wt. %, or about 0.1 to about 3 wt. % of one or more miscellaneous ingredients, including ranges and sub-ranges therebetween, based on the total weight of the composition.

### FURTHER EMBODIMENTS

In various embodiments, the compositions of the instant disclosure may optionally include one or more of polyethylene glycols (PEG), anionic surfactants, anionic polymers, or combinations thereof. In certain embodiments, the compositions include polyethylene glycol, for example, having at least 2, 3, 5, 10, or 100 repeating units. In other embodiments, the compositions are free or essentially free from polyethylene glycol, for example, polyethylene glycol having at least 2, 3, 10, or 100 repeating units. If present, the amount of polyethylene glycol may be from about 0.01 to about 10 wt. %, about 0.1 to about 5 wt. %, or about 0.1 to about 3 wt. %, including ranges and sub-ranges therebetween, based on the total weight of the compositions.

In various embodiments, the compositions of the instant disclosure are free or essentially free from anionic surfactants. In other embodiments, the compositions include one or more anionic surfactants. Anionic surfactants are known in the art. Nonlimiting examples include sulfate surfactants, isethionate surfactants, sarcosinate surfactants, sulfinate surfactants, taurate surfactants, etc. If present, the total amount of the one or more anionic surfactants may be from about 0.001 to about 5 wt. %, about 0.001 to about 3 wt. %, about 0.001 to about 1 wt. %, about 0.01 to about 4 wt. %, or about 0.1 to about 1 wt. %, based on the total weight of the composition. In other embodiments, the compositions of the instant disclosure are free or essentially free from anionic surfactants.

In various embodiments, the compositions of the instant disclosure are free or essentially free from anionic polymers. In other embodiments, however, the compositions include one or more anionic polymers. In the context of the present disclosure, the term “anionic polymers” is understood as meaning those polymers which carry in a protic solvent under standard conditions at least one structural unit having permanently anionic groups, the anionic groups having to be compensated by counterions while maintaining electroneutrality. As contemplated herein, anionic groups are, for example, carboxylate, sulfate, or sulfonate groups. Nonlimiting examples of anionic polymers include anionic polyurethanes, sodium polynaphthalene sulfonate, sodium lignosulfonate, sodium carboxymethyl cellulose, sodium salt of hydrophobically modified maleic anhydride copolymer, sodium polyacrylate, sodium polymethacrylate, ammonium polyacrylate, ammonium polymethacrylate, sodium salt of polymethacrylic acid, polystyrene sulfonate salts, carrageenan salts, dextran sulfate salts, polyacrylic acid salts, poly(methacrylic acid) salts, alginic acid salts, carboxymethylcellulose salts, polystyrene sulfonate/polystyrene copolymer salts, polystyrene sulfonate/maleic acid copolymers salt, copolymers thereof and combinations thereof.

In a preferred embodiment, the compositions of the instant disclosure are free or essentially free from anionic polymers. For example, the compositions may be free or essentially free from any one or more of the anionic polymers referenced above. In addition, the compositions of the instant disclosure may be free or essentially free from one or more, or all of the anionic polymers selected from anionic polyurethanes, sodium polynaphthalene sulfonate, sodium lignosulfonate, sodium carboxymethyl cellulose, sodium salt of hydrophobically modified maleic anhydride copolymer, sodium polyacrylate, sodium polymethacrylate, ammonium polyacrylate, ammonium polymethacrylate, sodium salt of polymethacrylic acid, polystyrene sulfonate salts, carrageenan salts, dextran sulfate salts, polyacrylic acid salts, poly(methacrylic acid) salts, alginic acid salts, carboxymethylcellulose salts, polystyrene sulfonate/polystyrene copolymer salts, polystyrene sulfonate/maleic acid copolymers salt, copolymers thereof and a combination thereof.

In various embodiments, the hair treatment compositions of the instant disclosure include one or more polyurethanes. However, in other embodiments, the hair treatment compositions are free or essentially free from polyurethanes.

### PREFERRED EMBODIMENTS

According to embodiments of the instant disclosure, the hair treatment composition preferably comprises, consists essentially of, or consists of:
(a) about 2 to about 9 wt. %, preferably about 2 to about 8 wt. %, more preferably about 3 to about 6 wt. % of one or more fatty alcohols, preferably wherein the one or more fatty alcohols are selected from decyl alcohol, undecyl alcohol, dodecyl, myristyl, cetyl alcohol, stearyl alcohol, cetearyl alcohol, isostearyl alcohol, isocetyl alcohol, behenyl alcohol, linalool, oleyl alcohol, myricyl alcohol and a combination thereof. In some instances, the cosmetic compositions preferably include cetyl alcohol, behenyl alcohol, cetearyl alcohol, or a combination thereof, more preferably cetearyl alcohol;
(b) 2 to 4 wt. % of one or more mono-alkyl cationic surfactants, preferably wherein the one or more mono-alkyl cationic surfactants are selected from mono-alkyl trimonium halide compounds, for example, one or more mono-alkyl trimonium halide compounds selected from cetrimonium chloride, steartrimonium chloride, behentrimonium chloride, cocotrimonium chloride, cocamidopropyltrimonium chloride, or a combination thereof;
(c) 0.1 to 3 wt. % of one or more di-alkyl cationic surfactants, preferably wherein the one or more di-alkyl cationic surfactants are selected from di-alkyl dimonium halide compounds, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, dicetyl dimethyl ammonium chloride, dicetyldimonium chloride, dicetyldimonium bromide, or a combination thereof;
   preferably, wherein (b) and (c) are in a weight ratio of about 2:1 to 10:1, preferably about 2:1 to about 6:1, more preferably about 3:1 to about 6:1 ((b):(c));
   preferably wherein (a) is in a weight ratio with (b)+(c) of about 1:1 to about 2.4:1, preferably about 1.2:1 to about 2.3:1, more preferably about 1.5:1 to about 2.2:1 ((a):((b)+(c));
(d) 1 to 10 wt. %, preferably about 1 to about 6 wt. %, more preferably about 1.5 to about 5 wt. % of one or more fatty compounds other than the fatty alcohols of (a),
   wherein at least one of the one or more fatty compounds is a fatty ester wax in an amount of at least 1 wt. %, preferably at least 1.1 wt. %, more preferably at least 1.2 wt. % based on the total weight of the hair treatment composition,
   wherein the at least one wax is preferably selected from hydrogenated castor oil wax, candelilla wax, carnauba wax, hydrogenated jojoba wax, beeswax, ozokerite, lanolin wax, montan wax, paraffin, palm kernel wax, ceresin, silicone waxes, cetyl esters, or a combination thereof;
(e) 0.5 to 10 wt. %, preferably about 1 to about 5 wt. %, more preferably about 1 to about 3 wt. % of one or more silicone oils, preferably wherein the one or more silicone oils are selected from dimethicone, dimethiconol, cyclomethicone, polysilicone-11, phenyl trimethicone, or a combination thereof;
(f) 0.1 to 10 wt. %, preferably about 0.5 to about 5 wt. %, more preferably about 0.5 to about 3 wt. % of one or more amino-functionalized silicones, preferably wherein the one or more amino-functionalized silicones are selected from amodimethicone, bis-hydroxy/methoxy amodimethicone, bis-cetearyl amodimethicone, bis(C13-15 alkoxy) PG amodimethicone, aminopropyl phenyl trimethicone, aminopropyl dimethicone, bis-amino PEG/PPG-41/3 aminoethyl PG-propyl dimethicone, or a combination thereof; and
(g) 60 to 90 wt. %, preferably about 65 to about 85 wt. %, more preferably about 70 to about 85 wt. % of water;
(h) optionally, about 0.1 to about 15 wt. %, preferably about 1 to about 8 wt. %, more preferably about 1 to about 8 wt. %, of one or more water-soluble solvents, preferably one or more water soluble solvents selected from glycerin, C₁-C₆ mono-alcohols, polyols (polyhydric alcohols), glycols, or a combination thereof;
(i) optionally, about 0.01 to about 5 wt. %, preferably about 0.05 to about 4 wt. %, more preferably about 0.05 to about 3 wt. of one or more thickening agents, preferably one or more thickening agents selected from nonionic polysaccharide thickening polymers selected from sclerotium gum, guar gums, guar gum derivatives, xanthan gum, hydroxyalkyl celluloses optionally modified with a hydrophobic group, inulins, carrageenans, carrageenan derivatives, or a combination thereof;
(j) optionally, up to about 5 wt. %, preferably about 0.01 to about 5 wt. %, more preferably about 0.05 to about 3 wt. % of one nonionic surfactants, amphoteric surfactants, or combination thereof; and
(k) optionally, about 0.1 to about 10 wt. %, preferably about 0.5 to about 8 wt. %, more preferably about 1 to about 5 wt. % of one or more miscellaneous ingredients selected from amino acids, pH adjusters, salts, fragrances, preservatives, antioxidants, composition colorants, botanical extracts, proteins, peptides, particulate fillers, vitamins, or a combination thereof;
   wherein all weight percentages are based on a total weight of the hair treatment composition.

According to a preferred embodiment, the hair treatment composition preferably comprises, consists essentially of, or consists of:
(a) about 2 to about 9 wt. %, preferably about 2 to about 8 wt. %, more preferably about 3 to about 6 wt. % of one or more fatty alcohols are selected from C14-22 linear fatty alcohols preferably cetearyl alcohol;
(b) 2 to 4 wt. % of cetrimonium chloride, behentrimonium chloride, or a combination thereof;
(c) 0.1 to 3 wt. % of one or more di-alkyl dimonium halide compounds selected from dicetyldimonium chloride, dicetyldimonium bromide, or a combination thereof;
   wherein (b) and (c) are in a weight ratio of about 2:1 to 10:1, preferably about 2:1 to about 6:1, more preferably about 3:1 to about 6:1 ((b):(c));
   wherein (a) is in a weight ratio with (b)+(c) of about 1:1 to about 2.4:1, preferably about 1.2:1 to about 2.3:1, more preferably about 1.5:1 to about 2.2:1 ((a):((b)+(c));
(d) 1 to 10 wt. %, preferably about 1 to about 6 wt. %, more preferably about 1.5 to about 5 wt. % of one or more fatty compounds other than the fatty alcohols of (a),
   wherein at least one of the one or more fatty compounds is a fatty ester wax in an amount of at least 1 wt. %, preferably at least 1.1 wt. %, more preferably at least 1.2 wt. % based on the total weight of the hair treatment composition,
   wherein the at least one the wax is selected from hydrogenated castor oil wax, candelilla wax, carnauba wax, hydrogenated jojoba wax, beeswax, ozokerite, lanolin wax, montan wax, paraffin, palm kernel wax, ceresin, silicone waxes, cetyl esters, or a combination thereof, preferably cetyl esters;
(e) 0.5 to 10 wt. %, preferably about 1 to about 5 wt. %, more preferably about 1 to about 3 wt. % of one or more silicone oils selected from dimethicone, dimethiconol, cyclomethicone, polysilicone-11, phenyl trimethicone, or a combination thereof, preferably dimethicone;
(f) 0.1 to 10 wt. %, preferably about 0.5 to about 5 wt. %, more preferably about 0.5 to about 3 wt. % of one or more amino-functionalized silicones selected from amodimethicone, bis-hydroxy/methoxy amodimethicone, bis-cetearyl amodimethicone, bis(C₁₃₋₁₅ alkoxy) PG amodimethicone, aminopropyl phenyl trimethicone, aminopropyl dimethicone, bis-amino PEG/PPG-41/3 aminoethyl PG-propyl dimethicone, or a combination thereof, preferably amodimethicone; and
(g) 60 to 90 wt. %, preferably about 65 to about 85 wt. %, more preferably about 70 to about 85 wt. % of water;
(h) optionally, about 0.1 to about 15 wt. %, preferably about 1 to about 8 wt. %, more preferably about 1 to about 8 wt. %, of one or more water-soluble solvents, preferably one or more water soluble solvents selected from glycerin, C₁-C₆ mono-alcohols, polyols (polyhydric alcohols), glycols, or a combination thereof;
(i) optionally, about 0.01 to about 5 wt. %, preferably about 0.05 to about 4 wt. %, more preferably about 0.05 to about 3 wt. of one or more thickening agents, preferably one or more thickening agents selected from nonionic polysaccharide thickening polymers selected from sclerotium gum, guar gums, guar gum derivatives, xanthan gum, hydroxyalkyl celluloses optionally modified with a hydrophobic group, inulins, carrageenans, carrageenan derivatives, or a combination thereof;
(j) optionally, up to about 5 wt. %, preferably about 0.01 to about 5 wt. %, more preferably about 0.05 to about 3 wt. % of one nonionic surfactants, amphoteric surfactants, or combination thereof; and
(k) optionally, about 0.1 to about 10 wt. %, preferably about 0.5 to about 8 wt. %, more preferably about 1 to about 5 wt. % of one or more miscellaneous ingredients selected from amino acids, pH adjusters, salts, fragrances, preservatives, antioxidants, composition colorants, botanical extracts, proteins, peptides, particulate fillers, vitamins, or a combination thereof;
   wherein all weight percentages are based on a total weight of the hair treatment composition.

### Viscosity

The compositions of the instant disclosure typically have a viscosity of about 1 mPa·s to about 1,000 mPa·s at 25° C., about 1 to about 500 mPa·s, or about 1 to about 100 mP·s. The viscosity measurements can be carried out, for example, using a Brooksfield viscometer, Model: DV−//+Pro (Brookfield Engineering Laboratories, Inc.) with spindle Helipath-94 and rotational speed of 12% RPM. As noted above, one or more thickening polymers may optionally be included in the compositions, which will increase the viscosity of the compositions.

In various embodiments, the composition have a viscosity from about 1 mPa·s to about 500 Pa·s, about 1 Pa·s to about 250 Pa·s, about 1 Pa·s to about 100 Pas, about 5 Pa·s to about 500 Pa·s, about 5 Pa·s to about 250 Pa·s, about 5 Pa·s to about 100 Pas, about 10 Pa·s to about 500 Pa·s, about 10 Pa·s to about 250 Pa·s, about 10 Pa·s to about 100 Pas, about 30 Pa·s to about 500 Pa·s, about 30 Pa·s to about 250 Pa·s, about 30 Pa·s to about 100 Pas, about 50 Pa·s to about 500 Pa·s, about 50 Pa·s to about 250 Pa·s, about 50 Pa·s to about 100 Pas, or about 50 Pa·s to about 75 Pa·s.

### Methods

The hair treatment compositions of the instant disclosure are useful for treating the hair, for example, for conditioning, managing, and improving the look and feel of hair. The compositions can be applied to the wet or damp hair and may be massaged into the hair, for example, with the hands, and/or spread throughout the hair with a comb or brush. This results in a smoothing and softening of the hair, which reduces frizz, dryness, and unwanted volume. The compositions can remain in the hair or can optionally be rinsed from the hair prior to drying and/or styling of the hair. Preferably, the hair treatment composition is rinsed from the hair after being applied and massaged into the hair.

The hair treatment compositions, in certain embodiments, may be used as a leave-on product. The hair treatment compositions are applied to wet or damp hair and allowed to remain on the hair indefinitely, i.e., the hair composition is not removed or rinsed from the hair prior to drying or styling the hair.

The methods of treating hair according to the disclosure include methods according to various routines. For instance, the compositions may be mixed with a shampoo (or conditioner) prior to application to the hair. Alternatively, the composition may be layered on top of (or lathered into) hair to which the shampoo (or conditioner) is already applied. Furthermore, the composition may be applied separate from the shampoo (or conditioner), i.e., applied to the hair after the shampoo (or conditioner) has been rinsed from the hair. In some instances, it is preferable to treat the hair with a composition of the instant disclosure prior to shampooing the hair, e.g., apply the composition to wet or damp hair prior to application of a shampoo to the hair. The hair may additionally (optionally) be treated with a conditioner after shampooing.

### Kits

The hair compositions of the instant disclosure may be incorporated into a kit. For example, the kits may include at least one hair treatment composition according to the instant disclosure and one or more additional compositions, for example, a shampoo, a conditioner, etc. The various compositions are separately contained in the kits. In some instances, the kits include one or more compositions according to the instant disclosure and a shampoo, both of which are separately contained. The kits may also include one or more compositions according to the instant disclosure, a shampoo, and a conditioner, all of which are separately contained. Instructions, mixing components, brushes, gloves, measuring tools, etc., may optionally be included in the kits.

The compositions may be packaged in a variety of different containers, such as, for example, a ready-to-use container. Non-limiting examples of useful packaging include tubes, jars, caps, unit dose packages, and bottles, including squeezable tubes and bottles. In some cases, the packaging is a tube, such as a tube with two compartments, or dual tubes, each forming a separate compartment. Each compartment may include a different composition. For example, one tube or compartment may include a composition according to the instant disclosure, and the other tube may include a composition to be used with the composition, for example, a shampoo, a conditioner, an all-in-one shampoo/conditioner (i.e., a conditioning shampoo; also referred to as a “co-wash”).

Methods of treating hair according to the disclosure will vary but typically include applying a hair treatment composition of the instant disclosure to wet or damp hair, allowing the composition to remain on the hair for a sufficient amount of time, and rinsing the compositions from the hair. The composition may be applied to the hair in a sequence with other compositions. For example, the compositions may be applied to the hair before shampooing the hair, after shampooing the hair, before conditioning the hair, and/or after conditioning the hair. The compositions, however, are not required to be used in a sequence.

The compositions of the instant disclosure may be allowed to remain on the hair for a minimum amount of time before being rinsed from the hair, but it is not necessary to allow the composition to remain on the hair. Conveniently, the compositions can be applied and allowed to remain on the hair for a period of time that is typical for regular shampooing and/or conditioning. For example, the composition (whether combined with another hair-treatment composition such as a shampoo or conditioner) may be applied to the hair and allowed to remain on the hair for a few seconds (1, 2, 3, or 5 seconds) up to about 1, about 2, about 5, about 10, about 15, about 20, about 25, or about 30 minutes.

The hair treatment composition may be applied to the hair immediately after or before the hair it treated with another composition (e.g., a shampoo and/or a conditioner). For example, the hair-treatment compositions may be applied to the hair within about 1, 2, 5, 10, or 20 minutes before or after a shampoo and/or a conditioner is applied to the hair.

The compositions of the instant disclosure are unique in their ability to provide hair with improved manageability, long-lasting style and frizz control, and smoothness. Accordingly, the instant disclosure relates to methods for treating hair, for example, for improving the manageability of hair, for imparting lasting style and frizz control, and for imparting smoothness. More specifically, the compositions may be used in methods for conditioning the hair, providing curl definition to the hair, providing frizz control to the hair, improving ease of combability and detangling, and providing smoothness.

### EXAMPLES

Various changes can be made in the above-described compositions and methods without departing from the scope of the invention. Accordingly, it is intended that all disclosure contained in the above description and in the examples given below, shall be interpreted as illustrative and not in a limiting sense.

Example 1

| | | | INV. | Comparative | | | | | | |
|---|---|---|---|---|---|---|---|---|---|---|
| | | | A | B | C | D | E | F | G | H |
| (a) | Fatty Alcohol | CETEARYL | 6 | | 6 | 6 | 6 | 6 | 6 | 12 |
| | | ALCOHOL | | | | | | | | |
| (b) | Mono-Alkyl- | CETRIMONIUM | 2.4 | 2.4 | 0.02 | 2.4 | 2.4 | 2.4 | 2.4 | 2.4 |
| | Cationic | CHLORIDE AND/OR | | | | | | | | |
| | Surfactants | BEHENTRIMONIUM | | | | | | | | |
| | | CHLORIDE | | | | | | | | |
| (c) | Di-Alkyl | DICETYLDIMONIUM | 0.7 | 0.7 | 0.7 | | 0.7 | 0.7 | 0.7 | 0.7 |
| | Cationic | CHLORIDE | | | | | | | | |
| | Surfactants | | | | | | | | | |
| Ratio of (b):(c) | | | 3.4:1 | 3.4:1 | 0.02:1 | — | 3.4:1 | 3.4:1 | 3.4:1 | 3.4:1 |
| Ratio of (a):((b) + (c)) | | | 1.9:1 | — | 8.6:1 | 2.5:1 | 1.9:1 | 1.9:1 | 1.9:1 | 3.9:1 |
| (d) | Fatty | CETYL ESTERS | 1.5 | 1.5 | 1.5 | 1.5 | | 1.5 | 1.5 | 1.5 |
| | Compounds & | ISOPROPYL | 0.3 | 0.3 | 0.3 | 0.3 | 0.3 | 0.3 | 0.3 | 0.3 |
| | Cetyl Esters | MYRISTATE and/or | | | | | | | | |
| | | SOYBEAN OIL | | | | | | | | |
| (e) | Silicone Oil | DIMETHICONE | 1.3 | 1.3 | 1.3 | 1.3 | 1.3 | 0.8 | 1.3 | 1.3 |
| (f) | Amino-Silicone | AMODIMETHICONE | 0.9 | 0.9 | 0.9 | 0.9 | 0.9 | 0.9 | 0.1 | 0.9 |
| (h) | Water-Soluble | GLYCERIN | 2 | 2 | 2 | 2 | 2 | 2 | 2 | 2 |
| | Solvent | PROPYLENE | 3.8 | 3.8 | 3.8 | 3.8 | 3.8 | 3.8 | 3.8 | 3.8 |
| | | GLYCOL and | | | | | | | | |
| | | ISOPROPYL | | | | | | | | |
| | | ALCOHOL | | | | | | | | |
| (i) | Thickening | HYDROXYPROPYL | 0.2 | 0.2 | 0.2 | 0.2 | 0.2 | 0.2 | 0.2 | 0.2 |
| | Polymer | GUAR | | | | | | | | |
| (j) | Amphoteric | COCO-BETAINE | 0.1 | 0.1 | 0.1 | 0.1 | 0.1 | 0.1 | 0.1 | 0.1 |
| | Surfactant | | | | | | | | | |
| | Nonionic | TRIDECETH-10, | 0.1 | 0.1 | 0.1 | 0.1 | 0.1 | 0.1 | 0.1 | 0.1 |
| | Surfactants | TRIDECETH-3, | | | | | | | | |
| | | PEG-100 | | | | | | | | |
| | | STEARATE, and/or | | | | | | | | |
| | | STEARETH-6 | | | | | | | | |
| | Nonionic | PEG/PPG-4/12 | 0.9 | 0.9 | 0.9 | 0.9 | 0.9 | 0.9 | 0.9 | 0.9 |
| | Silicone | DIMETHICONE | | | | | | | | |
| | Surfactant | | | | | | | | | |

| | | | | | | | | | |
|---|---|---|---|---|---|---|---|---|---|
| (k) | Amino acids, ¹ pH adjusters, salts, | ≤5 | ≤5 | ≤5 | ≤5 | ≤5 | ≤5 | ≤5 | ≤5 |
| misc. | fragrances, preservatives, antioxidants, | | | | | | | | |
| | chelating agents, compositions | | | | | | | | |
| | colorants, botanical extracts, etc. | | | | | | | | |
| (g) | WATER | QS | QS | QS | QS | QS | QS | QS | QS |

| | | | | | | | | |
|---|---|---|---|---|---|---|---|---|
| pH | 3.9 | NA³ | 4.2 | 3.9 | 4.0 | 3.8 | 3.1 | 4.1 |
| Viscosity² | 56 | NA³ | 35 | 27 | 32 | 28 | 28 | 75 |
| Stable | Yes | No | Yes | Yes | Yes | Yes | Yes | Yes |

¹ Glutamic Acid, Arginine, Serine, Etc., or a combination thereof.
²Pa · s at 25° C. measured with a Brookfield DV-LL+ Pro Viscometer using Spindle Helipath-94 and rotational speed of 12% RPM.
³Unable to measure accurate pH and viscosity due to instability.

### Example 2

Testing was carried out to determine how the compositions of Example 1 performed on human volunteers. Testing was carried out on a total of 3 volunteers. The hair of each volunteer was initially cleansed with a standard shampoo. After rinsing the shampoo from the hair, while the hair was still wet (damp), Inventive Composition A was applied to half of the head of hair. One of Comparative Compositions C—H was applied to the other half of the hair of the head. Comparative Composition B was not tested because it was not stable. Shortly after preparation, Comparative Composition B phase separated. Inventive Composition A and the Comparative Compositions C—H were applied to the damp hair and massaged throughout the hair, and then Inventive Composition A and the Comparative Compositions C—H remained on the hair for about 1 minute. The compositions then were rinsed from the hair. After rinsing was completed, the hair, while still wet, was independently evaluated by a panel of three expert hair evaluators, on a scale of 1 to 5 for the attributes listed in the table below. The hair was blow dried and again evaluated by the panel of three expert hair evaluators.
“1” Indicates the attribute was better than the Inventive Composition (benchmark).
“2” Indicates the attribute was slightly better than the Inventive Composition (benchmark).
“3” Indicates the attribute was equivalent to the Inventive Composition (benchmark) “4” Indicates the attribute was slightly inferior to the Inventive Composition (benchmark).
“5” Indicates the attribute was inferior to the Inventive Composition (benchmark).

The scores from the panels of expert evaluators were averages and are presented in the table below.

| Attributes | INV. | Comparative | | | | | | |
|---|---|---|---|---|---|---|---|---|
| Tested | A | B | C | D | E | F | G | H |
| Wet Combing | Benchmark | Not | 2 | 4 | 3 | 3 | 4 | 3 |
| Wet Smoothness | | Tested | 4 | 5 | 4 | 4 | 5 | 2 |
| Dry Combing | | (Phase | 3 | 4 | 3 | 4 | 3 | 3 |
| Dry Smoothness | | Separated) | 4 | 4 | 2 | 4 | 2 | 4 |
| Lightness | | | 3 | 4 | 5 | 3 | 4 | 4 |
| Anti-Frizz | | | 5 | 5 | 2 | 4 | 4 | 3 |

The results show that none of the Comparative Compositions achieved results comparable to those of Inventive Composition A.

As already mentioned, Comparative Composition B was unstable. This shows the need for one or more fatty alcohols in the composition. Despite containing other fatty compounds (cetyl esters, isopropyl myristate, and soybean oil), Comparative Composition B was unstable without the one or more fatty alcohols.

Comparative Composition C illustrates the need for a sufficient amount of the one or more mono-alkyl cationic surfactants (such as ceterimonium chloride and/or behentrimonium chloride). Comparative Composition C, which lacked a sufficient amount of the one or more mono-alkyl cationic surfactants failed to provide adequate smoothness and did not provide sufficient anti-frizz properties to the hair.

Comparative Composition D illustrates the need for one or more di-alkyl cationic surfactants (such as dicetyldimonium chloride). Comparative Composition D, which lacked di-alkyl cationic surfactants, failed to provide any attributes to the hair comparable with Inventive Composition A.

Comparative Composition E illustrates the need for one or more fatty ester waxes (such as cetyl esters). Comparative Composition D, which lacked a fatty ester wax, showed improvements to dry hair (smoothness and anti-frizz properties) but the hair had a “weighed down” appearance relative to Inventive Composition A. It also failed to provide improvements to wet hair.

Comparative Composition F illustrates the importance of the silicone oil (such as dimethicone). Absent sufficient silicone oil, Comparative Composition F failed to provide the desired amount of smoothness, ease of dry combing, and anti-frizz properties.

Comparative Composition G illustrates the importance of an amino-functionalized silicone (such as amodimethicone). Absent sufficient amino-functionalized silicone, Comparative Composition G failed to provide the desired degree of wet combing and smoothness and provided less lightness and anti-frizz attributes to the hair.

Comparative H include high amounts of one or more fatty alcohols (12 wt. %). Although treatment with Comparative Composition H showed a slight improvement with respect to wet smoothness, it failed to provide improvements to the dry hair. It performed worse than Inventive Composition A with respect to dry smoothness and lightness.

The terms “comprising,” “having,” and “including” are used in their open, non-limiting sense.

The terms “a” and “the” are understood to encompass the plural as well as the singular.

The term “cationic surfactant” as defined by the instant disclosure is a surfactant that may be positively charged when it is contained in the hair treatment compositions according to the disclosure. The cationic surfactant may bear one or more positive permanent charges or may contain one or more functional groups that are cationizable in the composition according to the disclosure.

The compositions and methods of the present disclosure can comprise, consist of, or consist essentially of the essential elements and limitations of the disclosure described herein, as well as any additional or optional ingredients, components, or limitations described herein or otherwise useful.

A “hair coloring agent” or “hair colorant” is a compound or ingredient included in compositions to intentionally change the color of hair. Nonlimiting examples of coloring agents include direct dyes, oxidative dye precursors, couplers, bleaching agents (e.g., peroxides such as hydrogen peroxide), etc.

A “hair coloring composition” is a composition designed and used to intentionally change the color of hair.

A “composition colorant” is a compound or ingredient that colors the composition but does not have an appreciable coloring effect on hair. In other words, the composition colorant is included to provide a color to the composition for aesthetic appeal but is not intended to impart coloring properties to hair. Styling gels, for example, can be found in a variety of different colors (e.g., light blue, light pink, etc.) yet application of the styling gel to hair does not visibly change the color of the hair. Thus, a composition colorant is different from a hair coloring agent. A composition colorant colors the compositions; a hair coloring agent may change the color of the compositions (and often does) but also changes the color of hair

The term “rinse-off” as used herein indicates that the composition is used in a context whereby the composition is ultimately rinsed or washed from the treated surface, (e.g. skin, hair, or hard surfaces) either after or during the application of the product. These rinse-off compositions are to be distinguished from compositions referred to a “leave-on” compositions. For example, a rinse-off composition is applied to the hair, optionally allowed to remain on the hair for a short time (e.g., a few second up to about 5, 10, or 15 minutes) and subsequently rinsed from the hair before the hair styled.

The compositions described throughout this disclosure may be a “leave-on” composition. A “leave-on” (also called leave-in) composition refers to a composition that is applied to skin or hair and is not subjected to immediate rinsing and/or washing, for example for at least 4 hours or for a period of time ranging from 4 hours up to 72 hours, from 4 hours up to 48 hours, or from 8 hours up to 36 hours, or from 8 hours up to 24 hours. In other words, the product is applied to the hair and remains on the hair, as styled, i.e., it is not removed from the hair prior to styling the hair.

A “lamellar phase” refers generally to packing of polar-headed long chain nonpolar-tail molecules in an environment of bulk polar liquid (i.e., water from the hair), as sheets of bilayers separated by bulk liquid.

The term “non-linear fatty acids” as used in the instant disclosure refers to unsaturated fatty acid and/or branched fatty acids.

The term “transparent” with respect to a transparent composition indicates that the composition has transmittance of at least 80% at a wavelength of 600 nm, for example measured using a Lambda 40 UV-visible spectrometer. The compositions may have, for example, a transmittance of at least 80%, at least 90%, or at least 95% at a wavelength of 600 nm, measured, for example, using a Lambda 40 UV-visible spectrometer. The term “clear” is interchangeable with the term “transparent” for purposes of the instant disclosure. A human can typically see through a transparent composition, for example, and read the text on the other side of a clear glass or clear plastic bottle containing the composition.

The term “translucent” with respect to a translucent composition indicates that the composition has a transmittance of at least 50% at a wavelength of 600 nm, for example measured using a Lambda 40 UV-visible spectrometer. A human cannot likely see through a translucent composition, for example, and read the text on the other side of a clear glass or clear plastic bottle containing the composition. Rather, the text is usually blurred and difficult or not possible to read, yet movement and structure can normally be identified.

The term “opaque” with respect to an opaque composition indicates that the composition is not transparent or translucent, i.e., has a transmittance of less than 50% at a wavelength of 600 nm, for example measured using a Lambda 40 UV-visible spectrometer.

All percentages, parts and ratios herein are based upon the total weight of the compositions of the present disclosure, unless otherwise indicated.

All ranges and values disclosed herein are inclusive and combinable. For examples, any value or point described herein that falls within a range described herein can serve as a minimum or maximum value to derive a sub-range, etc. Furthermore, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

Other than in the operating examples, or where otherwise indicated, all numbers expressing quantities of ingredients and/or reaction conditions are to be understood as being modified in all instances by the term “about,” meaning within +/−5% of the indicated number.

As used herein, the expression “at least one” is interchangeable with the expression “one or more” and thus includes individual components as well as mixtures/combinations.

The term “substantially free” or “essentially free” as used herein means that there is at least less than about 5% by weight of a specific material added to a composition, based on the total weight of the compositions. Nonetheless, the compositions may include less than about 2 wt. %, less than about 1 wt. %, less than about 0.5 wt. %, less than about 0.1 wt. %, less than 0.01 wt. %, or none of the specified material.

The term “active material” as used herein with respect to the percent amount of an ingredient or raw material, refers to 100% activity of the ingredient or raw material. All ingredients are listed as 100% active material unless otherwise indicated.

“Cosmetically acceptable” means that the item in question is compatible with a keratinous substrate such as skin and hair. For example, a “cosmetically acceptable carrier” means a carrier that is compatible with a keratinous substrate such as skin and hair.

Throughout the disclosure, the term “a combination thereof” (or a mixture thereof) may be used following a list of elements as shown in the following example where letters A-F represent the elements: “one or more elements selected from the group consisting of A, B, C, D, E, F, or a combination thereof.” The term, “a combination thereof” does not require that the combination include all of A, B, C, D, E, and F (although all of A, B, C, D, E, and F may be included). Rather, it indicates that a combination of any two or more of A, B, C, D, E, and F can be included. In other words, it is like the phrase “one or more elements selected from the group consisting of A, B, C, D, E, F, and a combination of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, or a combination thereof,” it indicates that that one or more of A, B, C, D, and F may be included, one or more of a salt of A, a salt of B, a salt of C, a salt of D, a salt of E, and a salt of F may be included, or a combination of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counter-ion. This list of counter-ions, however, is non-limiting.

Some of the compounds discussed throughout the disclosure may be in the form of a salt in the composition or added to the composition in the form of a salt (and dissociate in the composition). Thus, all compounds and amounts of compounds relate to both the salt form of the compound and to the disassociated form of the compound. In other words, even if the expression “a salt thereof” is not specifically or expressly stated with respect to ingredients that can form salts or are available as salts, it is understood that the salt form of the compound is included.

The expression “inclusive” for a range of concentrations means that the limits of the range are included in the defined interval.

“Volatile”, as used herein, means having a flash point of less than about 100° C.

“Non-volatile”, as used herein, means having a flash point of greater than about 100° C.

The term “polymers,” as defined herein, include homopolymers and copolymers formed from at least two different types of monomers.

The term “INCI” is an abbreviation of International Nomenclature of Cosmetic Ingredients, which is a system of names provided by the International Nomenclature Committee of the Personal Care Products Council to describe personal care ingredients.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

All components and elements positively set forth in this disclosure can be negatively excluded from the claims. In other words, the compositions of the instant disclosure can be free or essentially free of any components and elements positively recited throughout the instant disclosure.

Some of the various categories of components identified may overlap. In such cases where overlap may exist and the composition includes both components (or the composition includes more than two components that overlap), an overlapping component or ingredient does not represent more than one component of the claims. For example, if a polyacrylate falls within the description of a thickening polymer and within the description of film forming polymer, a single polyacrylate is understood as being only the thickening polymer or only the film forming polymer. A single polyacrylate cannot simultaneously be construed as both a thickening polymer and a film forming polymer, for example, when the claims set forth both a thickening polymer and a film forming polymer (even though the polyacrylate may function as both a thickening polymer and a film forming polymer). In other words, a single compound or ingredient cannot simultaneously serve as two different components or elements of a claim.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A hair treatment composition comprising:
(a) 2 to 10 wt. % of one or more fatty alcohols
(b) 2 to 4 wt. % of one or more mono-alkyl cationic surfactants;
(c) 0.1 to 3 wt. % of one or more di-alkyl cationic surfactants;
(d) 1 to 10 wt. % of one or more fatty compounds other than the fatty alcohols of (a),
wherein at least one of the one more fatty compounds is a fatty ester wax in an amount of at least 1 wt. %, based on a total weight of the hair treatment composition;
(e) 1 to 10 wt. % of one or more silicone oils; and
(f) 0.5 to 10 wt. % of one or more amino-functionalized silicones; and
(g) 60 to 90 wt. % water,
wherein all weight percentages are based on the total weight of the hair treatment composition.

2. The hair treatment composition of claim 1, wherein the one or more fatty alcohols are selected from decyl alcohol, undecyl alcohol, dodecyl, myristyl, cetyl alcohol, stearyl alcohol, cetearyl alcohol, isostearyl alcohol, isocetyl alcohol, behenyl alcohol, linalool, oleyl alcohol, myricyl alcohol, and a combination thereof.

3. The composition of claim 1, wherein the one or more fatty alcohols are selected from C14-22 linear fatty alcohols.

4. The hair treatment composition of claim 1, wherein the one or more mono-alkyl cationic surfactants are mono-alkyl trimonium halide compounds selected from cetrimonium chloride, steartrimonium chloride, behentrimonium chloride, cocotrimonium chloride, cocamidopropyltrimonium chloride, or a combination thereof.

5. The hair treatment composition of claim 1, wherein the one or more di-alkyl cationic surfactants are di-alkyl dimonium halide compounds selected from dialkyl (14-18) dimethyl ammonium chloride, ditallow alkyl dimethyl ammonium chloride, dihydrogenated tallow alkyl dimethyl ammonium chloride, distearyl dimethyl ammonium chloride, dicetyl dimethyl ammonium chloride, dicetyldimonium chloride, dicetyldimonium bromide, or a combination thereof.

6. The hair treatment composition of claim 1, wherein:
(b) and (c) are in a weight ratio of 2:1 to 8:1 ((b):(c)), and/or
(a) is in a weight ratio with (b) and (c) of 1:1 to 2.4:1 ((a):((b)+(c)).

7. The hair treatment composition of claim 1, wherein the one or more fatty ester wax is selected from hydrogenated castor oil wax, candelilla wax, carnauba wax, hydrogenated jojoba wax, beeswax, ozokerite, lanolin wax, montan wax, paraffin, palm kernel wax, ceresin, silicone waxes, cetyl esters, or a combination thereof.

8. The hair treatment composition of claim 1, wherein the one or more silicone oils are selected from dimethicone, dimethiconol, cyclomethicone, polysilicone-11, phenyl trimethicone, or a combination thereof.

9. The hair treatment composition of claim 1, wherein the one or more amino-functionalized silicones are selected from amodimethicone, bis-hydroxy/methoxy amodimethicone, bis-cetearyl amodimethicone, bis(C13-15 alkoxy) PG amodimethicone, aminopropyl phenyl trimethicone, aminopropyl dimethicone, bis-amino PEG/PPG-41/3 aminoethyl PG-propyl dimethicone, or a combination thereof.

10. The hair treatment composition of claim 1, wherein at least one of the one or more silicone oils is dimethicone and at least one of the one or more amino-functionalized silicones is amodimethicone.

11. The hair treatment composition of claim 1, further comprising:
(h) about 1 to about 15 wt. % of one or more water-soluble solvents selected from glycerin, C₁-C₆ mono-alcohols, polyhydric alcohols, glycols, or a combination thereof.

12. The hair treatment composition of claim 1, wherein the hair treatment composition further comprises:
(i) 0.1 to 5 wt. % of one or more thickening agents.

13. The hair treatment composition of claim 12, wherein the one or more thickening agents include one or more nonionic polysaccharide thickening polymers selected from sclerotium gum, guar gums, guar gum derivatives, xanthan gum, hydroxyalkyl celluloses optionally modified with a hydrophobic group, inulins, carrageenans, carrageenan derivatives, or a combination thereof.

14. The hair treatment composition of claim 1, further comprising:
(j) one or more amphoteric surfactants, nonionic surfactants, cationic surfactants other than the one or more mono-alkyl cationic surfactants (b) and the one or more di-alkyl cationic surfactants of (c), or a combination thereof.

15. The hair treatment composition of claim 1, further comprising:
(k) one or more miscellaneous ingredients selected from amino acids, pH adjusters, salts, fragrances, preservatives, antioxidants, composition colorants, botanical extracts, proteins, peptides, particulate fillers, vitamins, or a combination thereof.

16. The hair treatment composition of claim 1 comprising:
(a) 3 to 8 wt. % of one or more fatty alcohols
(b) 2 to 5 wt. % of one or more mono-alkyl ammonium halide;
(c) 0.1 to 3 wt. % of one or more di-alkyl ammonium halide,
wherein (b) and (c) are in a weight ratio of 8:1 to 2:1;
(d) 1 to 10 wt. % of one or more fatty compounds other than the fatty alcohols of (a),
wherein at least one of the one more fatty compounds is a fatty ester wax in an amount of at least 1 wt. %, based on a total weight of the hair treatment composition;
(e) 0.5 to 5 wt. % of one or more silicone oils;
(f) 0.1 to 5 wt. % of one or more amino-functionalized silicones;
(g) 60 to 85 wt. % of water;
(h) 0.1 to 15 wt. % of one or more water-soluble solvents;
(i) 0.1 to 3 wt. % of one or more thickening polymers;
(j) up to 5 wt. % of one or more amphoteric surfactants, nonionic surfactants, or a combination thereof; and
(k) up to 5 wt. % of one or more miscellaneous ingredients selected from amino acids, pH adjusters, salts, fragrances, preservatives, antioxidants, composition colorants, botanical extracts, proteins, peptides, particulate fillers, vitamins, or a combination thereof.

17. The hair treatment composition of claim 1 consisting essentially of:
(a) 3 to 8 wt. % of one or more C14-22 linear fatty alcohols;
(b) 2 to 5 wt. % of cetrimonium chloride, behentrimonium chloride, or a combination thereof;
(c) 0.1 to 3 wt. % of dicetyldimonium chloride,
wherein (b) and (c) are in a weight ratio of 6:1 to 3:1;
(d) 1 to 10 wt. % of one or more fatty compounds other than the fatty alcohols of (a),
wherein the at least one wax is cetyl esters;
(e) 1 to 3 wt. % of dimethicone, dimethiconol, cyclomethicone, phenyl trimethicone, or a combination thereof;
(f) 0.5 to 5 wt. % of amodimethicone;
(g) 70 to 85 wt. % of water;
(h) 1 to 10 wt. % of one or more water-soluble solvents;
(i) 0.1 to 3 wt. % of one or more nonionic polysaccharide thickening polymers;
(j) up to 5 wt. % of one or more amphoteric surfactants, nonionic surfactants, cationic surfactants other than the one or more mono-alkyl cationic surfactants (b) and the one or more di-alkyl cationic surfactants of (c), or a combination thereof; and
(k) up to 5 wt. % of one or more miscellaneous ingredients selected from amino acids, pH adjusters, salts, fragrances, preservatives, antioxidants, composition colorants, botanical extracts, proteins, peptides, particulate fillers, vitamins, or a combination thereof.

18. A kit comprising:
(i) a shampoo composition; and
(ii) a hair treatment composition of claim 1;
wherein the shampoo and the hair treatment composition are separately contained.

19. A method for treating hair comprising applying the hair treatment composition of claim 1 to the hair and optionally rinsing the hair treatment composition from the hair.

20. A method of claim 19, wherein the method provides frizz control to the hair; improves hair manageability; conditions the hair; preserves color of artificially colored hair; and/or improves hair fiber alignment.
